(19) United States
(12) Patent Application Publication
(10) Pub. No.: US 2022/0110546 A1
(43) Pub. Date: Apr. 14, 2022

(54) SYSTEM AND METHODS FOR TRACKING BEHAVIOR AND DETECTING ABNORMALITIES

(71) Applicants: EMORY UNIVERSITY, Atlanta, GA (US); GEORGIA TECH RESEARCH CORPORATION, Atlanta, GA (US)

(72) Inventors: Gari Clifford, Atlanta, GA (US); Jacob Zelko, Atlanta, GA (US); Nicolas Shu, Atlanta, GA (US); Pradyumna Suresha, Atlanta, GA (US); Ayse Cakmak, Atlanta, GA (US)

(73) Assignees: EMORY UNIVERSITY, Atlanta, GA (US); GEORGIA TECH RESEARCH CORPORATION, Atlanta, GA (US)

(21) Appl. No.: 17/430,414

(22) PCT Filed: Feb. 27, 2020
(86) PCT No.: PCT/US2020/020153

§ 371 Date: Aug. 12, 2021

Related U.S. Application Data
(60) Provisional application No. 62/811,266, filed on Feb. 27, 2019.

(51) Int. Cl. A61B 5/11; A61B 5/00; G16H 50/20

(52) U.S. Cl. CPC A61B 5/1118; A61B 5/1113; A61B 5/6889; G16H 50/20; A61B 5/7267; A61B 5/0022; A61B 5/7225; A61B 5/4818

(57) ABSTRACT

A computer-implemented method includes obtaining movement data associated with a subject and measured by a stationary motion sensor. The movement data includes a series of values representing a series of movement events of the subject crossing fields of view of the stationary motion sensor. Each movement event in the series of movement events is associated with a respective time stamp. The computer-implemented method further includes extracting a plurality of features from the movement data, determining that the movement data is consistent with symptoms of an illness using a machine-learning model and based upon the plurality of features, and generating an output indicating a result of the determination.

### CROSS-REFERENCE TO RELATED APPLICATION

This patent application claims benefit of and priority to U.S. Provisional Patent Application Ser. No. 62/811,266, filed Feb. 27, 2019, entitled “SYSTEM AND METHODS FOR TRACKING BEHAVIOR AND DETECTING ABNORMALITIES,” which is hereby incorporated by reference in its entirety for all purposes.

### TECHNICAL FIELD

Techniques disclosed herein relate generally to detecting abnormal behaviors indicative a certain illness. More specifically, one or more off-body motion sensors may measure movement events of a subject over a time period, the movement event data may then be analyzed to extract various features characterizing behaviors of the subject, and the extracted features may be used by a machine-learning-based classifier to determine abnormal behaviors of the subject that may be indicate a certain illness, such as obstructive sleep apnea or a mental illness.

### BACKGROUND

Obstructive sleep apnea is a common and serious sleep disorder in humans. Obstructive sleep apnea may affect oxygenation due to upper airway collapse, and thus may lead to, for example, excessive daytime sleepiness and long-term health complications, such as cardiovascular diseases, stroke, and abnormal glucose metabolism (e.g., type II diabetes). Symptoms of obstructive sleep apnea include, for example, repeated arousal and gasping for air during sleep. Obstructive sleep apnea may be particularly prevalent (e.g., about 3% to about 7%) among adult male population. However, the disease is often not diagnosed because it can be clinically silent. Further, widespread screening of asymptomatic individuals may not be recommended due to, for example, the cost and inconvenience associated with existing diagnostic techniques. For example, diagnosing sleep apnea based on clinical symptoms and polysomnography may include attaching multiple sensors to a subject to record various physiological signals during an overnight stay in a sleep lab. In addition to the associated cost, inconvenience, and time for analyzing the physiological signals, this arrangement positions the subject in an unnatural sleep environment that may produce data that is not typical for the subject.

### SUMMARY

Techniques disclosed herein relate generally to detecting abnormal behaviors of a subject that may indicate a certain illness using machine-learning models and coarse movement data. More specifically, one or more off-body motion sensors may be used to continuously measure movement events of the subject over a time period, and the movement event data may then be analyzed to extract certain features for use by one or more machine-learning-based classifiers to determine whether the subject is afflicted with an illness.

According to certain embodiments, a computer-implemented method may include obtaining movement data associated with a subject and measured by a stationary motion sensor. The movement data may include a series of values representing a series of movement events of the subject crossing fields of view of the stationary motion sensor. Each movement event in the series of movement events may be associated with a respective time stamp. The computer-implemented method may further include extracting a plurality of features from the movement data, determining that the movement data is consistent with symptoms of an illness using a machine-learning model and based upon the plurality of features, and generating an output indicating a result of the determination.

In some embodiments of the computer-implemented method, extracting the plurality of features from the movement data may include generating, based on the time stamps, activity data including, for each pair of adjacent movement events in the series of movement events, a respective inverse value of a time interval between the pair of adjacent movement events, and extracting a set of features from the activity data, where the plurality of features may include the set of features extracted from the activity data. In some embodiments, the set of features may include at least one of a shape or a scale of a Generalized Pareto Distribution of the activity data. In some embodiments, the set of features may include at least one of multiscale entropies for a plurality of different time scales of the activity data, a mean of the multiscale entropies, or a variance of the multiscale entropies.

In some embodiments, extracting the plurality of features from the movement data may include generating, based on the time stamps, time difference data indicative time intervals between adjacent movement events in the series of movement events, and computing a set of statistical parameters of the time difference data, where the plurality of features includes the set of statistical parameters of the time difference data. The set of statistical parameters of the time difference data may include at least one of a mean, variance, skewness, kurtosis, or interquartile range of the time difference data.

In some embodiments, determining that the movement data is consistent with the symptoms of the illness includes at least one of normalizing the plurality of features, performing a forward feature selection-based classification, or estimating an illness severity value or a confidence level of the determination. The machine-learning model may include one or more binary classifiers, such as a logistic regression model.

In some embodiments, the stationary motion sensor may include a passive infrared sensor. In some embodiments, the passive infrared sensor may include a pyroelectric infrared sensor. In some embodiments, the movement data may be collected at a pre-set sampling frequency, such as 1 Hz.

According to certain embodiments, a computer-program product tangibly embodied in a non-transitory machine-readable storage medium, may include instructions configured to cause one or more data processors to perform operations. The operations may include obtaining movement data associated with a subject and measured by a stationary motion sensor. The movement data may include a series of values representing a series of movement events of the subject crossing fields of view of the stationary motion sensor. Each movement event in the series of movement events may be associated with a respective time stamp. The computer-implemented method may further include extracting a plurality of features from the movement data, determining that the movement data is consistent with symptoms of an illness using a machine-learning model and based upon the plurality of features, and generating an output indicating a result of the determination.

In some embodiments, extracting the plurality of features from the movement data may include generating, based on the time stamps, activity data including, for each pair of adjacent movement events in the series of movement events, a respective inverse value of a time interval between the pair of adjacent movement events, and extracting a set of features from the activity data, where the plurality of features may include the set of features extracted from the activity data. In some embodiments, the set of features may include at least one of a shape of a Generalized Pareto Distribution of the activity data, a scale of the Generalized Pareto Distribution of the activity data, multiscale entropies for a plurality of different time scales of the activity data, a mean of the multiscale entropies, or a variance of the multiscale entropies.

In some embodiments, extracting the plurality of features from the movement data may include generating, based on the time stamps, time difference data indicative of time intervals between adjacent movement events in the series of movement events, and computing a set of statistical parameters of the time difference data, where the plurality of features includes the set of statistical parameters of the time difference data. The set of statistical parameters of the time difference data may include at least one of a mean, variance, skewness, kurtosis, or interquartile range of the time difference data.

According to certain embodiments, a system may include one or more data processors, and a non-transitory computer-readable storage medium containing instructions which, when executed on the one or more data processors, cause the one or more data processors to perform operations. The operations may include obtaining movement data associated with a subject and measured by a stationary motion sensor. The movement data may include a series of values representing a series of movement events of the subject crossing fields of view of the stationary motion sensor. Each movement event in the series of movement events may be associated with a respective time stamp. The computer-implemented method may further include extracting a plurality of features from the movement data, determining that the movement data is consistent with symptoms of an illness using a machine-learning model and based upon the plurality of features, and generating an output indicating a result of the determination.

In some embodiments, extracting the plurality of features from the movement data may include generating, based on the time stamps, activity data including, for each pair of adjacent movement events in the series of movement events, a respective inverse value of a time interval between the pair of adjacent movement events, and extracting a set of features from the activity data, where the plurality of features may include the set of features extracted from the activity data. In some embodiments, the set of features may include at least one of a shape of a Generalized Pareto Distribution of the activity data, a scale of the Generalized Pareto Distribution of the activity data, multiscale entropies for a plurality of different time scales of the activity data, a mean of the multiscale entropies, or a variance of the multiscale entropies.

In some embodiments, extracting the plurality of features from the movement data may include generating, based on the time stamps, time difference data indicative time intervals between adjacent movement events in the series of movement events, and computing a set of statistical parameters of the time difference data, where the plurality of features includes the set of statistical parameters of the time difference data. The set of statistical parameters of the time difference data may include at least one of a mean, variance, skewness, kurtosis, or interquartile range of the time difference data.

Many benefits can be achieved by techniques disclosed herein over conventional techniques. For example, the motion sensors may include low-cost motion sensors, such as a passive (e.g., pyroelectric) infrared sensor. The passive infrared sensor can generate binary or continuous signals or impulses indicating movements of a subject crossing different fields of view of the motion sensor, rather than information that may compromise the privacy of the subject or may use a large storage space or communication bandwidth, such as videos or images. Unlike a camera, the passive infrared sensor does not need to be accurately focused on the subject in order to detect motions of the subject. Furthermore, the low-cost motion sensors can be set up at one or more locations of structures frequented by the subject, such as a living room or a bedroom, rather than being worn by the subject, thereby facilitating convenient, non-invasive, and continuous measurement and monitoring of the health condition of the subject in a natural environment over a long period of time. In addition, the machine-learning-based classifiers can automatically extract various statistical, time-domain, and/or frequency-domain features from the measured movement data, can select features that can provide the best classification sensitivity and specificity, and can generate classification results quickly. Therefore, the classification can be made with little or no time delay and human intervention on or close to the sensor, or in a remote location with a low communication bandwidth. Moreover, the relevant features may be pre-selected and the machine-learning-based classifiers can be pre-trained and less complex (e.g., light weight), such that the classifiers may use little processing power and small memory space for storing parameters of the classifiers (e.g., the weights), and thus may be implemented using devices with low processing power and small memory space, such as some mobile devices and embedded systems.

This summary is neither intended to identify key or essential features of the claimed subject matter, nor is it intended to be used in isolation to determine the scope of the claimed subject matter. The subject matter should be understood by reference to appropriate portions of the entire specification of this disclosure, any or all drawings, and each claim. The foregoing, together with other features and examples, will be described in more detail below in the following specification, claims, and accompanying drawings.

### BRIEF DESCRIPTION OF THE DRAWINGS

Illustrative embodiments are described in detail below with reference to the following figures.

FIG. 1 illustrates an example of a system for detecting abnormal behaviors indicative a certain illness based on movement event measurement according to certain embodiments.

FIG. 2A illustrates an example of a motion sensor for detecting movement events of a subject according to certain embodiments.

FIG. 2B illustrates an example of motion detection based on heat source movements according to certain embodiments.

FIG. 2C illustrates an example of motion sensor output signals indicative movement events of a subject according to certain embodiments.

FIG. 3A is a side view of an example of a passive infrared sensor for detecting movement events of a subject according to certain embodiments.

FIG. 3B is a top view of an example of a passive infrared sensor for detecting movement events of a subject according to certain embodiments.

FIG. 4A illustrates an example of a passive infrared sensor that includes two infrared sensitive slots for detecting movement events of a subject according to certain embodiments.

FIG. 4B illustrates a simplified block diagram of an example of a passive infrared sensor for detecting movement events of a subject according to certain embodiments.

FIG. 5 is a flow chart illustrating an example of a method of detecting abnormal behaviors indicative a certain illness based on movement data according to certain embodiments.

FIG. 6 illustrates an example of pre-processing measured movement data according to certain embodiments.

FIG. 7 is a flow chart illustrating an example of a method of detecting abnormal behaviors indicative a certain illness based on pre-processed movement data according to certain embodiments.

FIG. 8 illustrates an example of generating multiscale time series using measured movement data according to certain embodiments.

FIG. 9 illustrates an example of forward feature selection-based classification using a leave-one-out cross-validation method according to certain embodiments.

FIG. 10A is a side view of another example of a system for detecting abnormal behaviors indicative a certain mental illness based on movement event measurement according to certain embodiments.

FIG. 10B is a top view of the example of the system of FIG. 10A for detecting abnormal behaviors indicative a certain mental illness based on movement event measurement according to certain embodiments.

FIG. 11 illustrates another example of a system for predicting negative behaviors based on movement event measurement according to certain embodiments.

FIG. 12A illustrates an example of activity data including inverse values of time intervals between detected movement events of a subject according to certain embodiments.

FIG. 12B illustrates an example of statistic data including numbers of movement events of the subject during different time periods according to certain embodiments.

FIG. 13A illustrates an example of training and test accuracies using Generalized Pareto Distribution (GPD) features extracted from movement data for one night according to certain embodiments.

FIG. 13B illustrates an example of training and test accuracies using GPD features extracted from movement data for two nights according to certain embodiments.

FIG. 13C illustrates an example of training and test accuracies using GPD features extracted from movement data for three nights according to certain embodiments.

FIG. 13D illustrates an example of training and test accuracies using GPD features extracted from movement data for four nights according to certain embodiments.

FIG. 14 is a simplified block diagram of an example of a computing system for implementing certain embodiments disclosed herein.

The figures depict embodiments of the present disclosure for purposes of illustration only. One skilled in the art will readily recognize from the following description that alternative embodiments of the structures and methods illustrated may be employed without departing from the principles, or benefits touted, of this disclosure.

In the appended figures, similar components and/or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

Techniques disclosed herein relate generally to detecting abnormal behaviors of a subject that may indicate a certain illness using movement data and machine-learning-based classifiers. More specifically, off-body motion sensors may be used to continuously measure privacy-preserving movement events of the subject over a time period, and movement data including information of the measured movement events may be analyzed to extract certain time-domain and frequency-domain features for use by one or more machine-learning-based classifiers to automatically determine whether the subject is afflicted with a certain illness. Various inventive embodiments are described herein, including systems, methods, devices, modules, models, algorithms, networks, structures, processes, computer-program products, and the like.

Subjects with some sleep disorders or mental illnesses may manifest abnormal activities at night (e.g., during sleep) or during daytime. The abnormal activities or behaviors may sometimes be clinically silent and thus may be difficult to detect for diagnosing illnesses. For example, obstructive sleep apnea is a sleep disorder that may cause people to temporarily stop breathing multiple times during the night, and may cause excessive daytime sleepiness and various long term health complications. Obstructive sleep apnea is typically diagnosed using polysomnography (PSG) in a sleep lab or using some wearable sleep monitors. The polysomnography or wearable sleep monitors may use multiple sensors attached to the subject's body to record various physiological signals during an overnight stay in the sleep lab, which may not represent the subject's natural sleep environment, in addition to the associated cost, inconvenience, and time for analyzing the physiological signals by physicians. The serious adverse effects of obstructive sleep apnea and the prevalence but under-diagnosis of obstructive sleep apnea have resulted in a growing demand for low cost, convenient, rapid, and accurate diagnosis of obstructive sleep apnea. In addition, obstructive sleep apnea is a chronic disorder, and thus lifelong at-home monitoring and care may be desired. At-home monitoring may also be desired in order to provide useful information regarding, for example, response to therapy and disease progression.

As another example, subjects with certain mental illnesses, such as anxiety disorders, major depression, bipolar disorders, and schizophrenia, may sometimes behave abnormally before a mental breakdown or a medical emergency occurs. In yet another example, there may be some correlations between the lack of sleep at night and certain short-term or long-term negative behaviors. Thus, it would be advantageous to identify a technique to improve the detectability of sleep abnormalities and/or associated conditions.

Some techniques can be used to detect abnormal behaviors indicative certain illnesses (e.g., obstructive sleep apnea) by monitoring behaviors of subjects. For example, physiological signals (e.g., vital signals) and/or visual, audio, or video data may be captured by wearable devices, cameras, or audio recorders. This data may then be used alone or in combination to detect abnormal behaviors. However, these techniques may involve a large amount of measurement data, which may also compromise the privacy of the subjects being monitored. The large amount of measurement data may use a large memory space for storage, a high bus bandwidth for data transferring, and a large amount of computation for processing and analysis. Therefore, these techniques may not be suitable for long time monitoring and/or real-time detection or classification of abnormal behaviors.

According to certain embodiments, a low-cost, passive, off-body sensor may be used to continuously monitor a subject in a privacy preserving manner for detecting abnormal behaviors of the subject that may indicate certain illnesses. The off-body sensor can measure movement events of the subject, where each measured movement event of the subject may be represented by, for example, an impulse or a binary signal (e.g., a high or low signal level) in a time series. In one example, the off-body sensor may include a passive infrared sensor, such as a pyroelectric infrared sensor, that can generate binary signals or impulses indicating movements of a subject crossing different fields of view of the passive infrared sensor based on changes in the amount of infrared radiation received by two infrared-sensitive slots of the passive infrared sensor. The off-body sensor may be positioned (e.g., secured or mounted) at one or more locations of structures frequented by the subject, such as a bedroom, rather than being worn by the subject, thereby facilitating convenient, non-invasive, and continuous measurement and monitoring of the health condition of the subject in a natural environment over a long period of time.

The movement data measured by the off-body sensor during a monitoring period, such as one or more nights, may be processed to generate time-domain and/or frequency-domain data, such as data characterizing and/or being based on: the time intervals between consecutive movement events, the numbers of movement events detected during a given time period, and the frequency (e.g., inverses of the time intervals) of the movement events during one or more time periods. Statistical and entropy-based features (such as Generalized Pareto Distribution (GPD) parameters, multiscale entropies (MSEs) in a plurality of different time scales, and other statistical parameters characterizing the movement data) may be extracted from the time-domain and/or frequency-domain signals and may be input to a machine-learning model for prediction or classification. In some embodiments, the machine-learning model may also perform the feature extraction.

The machine-learning model may include one or more classifiers that include (and/or implement and/or are based on), for example, logistic regression, support vector machines, decision trees, nearest neighbors, Bayes classifiers, and the like. Based on a feature vector that includes the statistical and/or entropy-based features extracted from the movement data, the machine-learning model may predict whether the subject is afflicted with a certain illness or manifests abnormal behaviors that may need attentions or intervention. In some embodiments, the machine-learning model may also determine the confidence level of the classification or the severity of the illness.

In some embodiments, forward feature selection techniques may be used to determine the features that can provide the best combination of classification sensitivity and specificity, such that only these features may be extracted and used to more quickly determine classification results. As such, the machine-learning model may be less complex and may use less processing power and less memory space for storing parameters of the model, and thus may be implemented using devices with low processing power and small memory space, such as some mobile devices (e.g., smart phones).

Therefore, techniques disclosed herein may be applied to a large population with lower cost, higher convenience, and faster classification speed than existing techniques. In addition, techniques disclosed herein may be non-invasive and privacy preserving, and may enable continuous monitoring of the health condition of subjects during daily activities in a more natural environment over a long period of time, thereby facilitating early diagnosis and treatment of illnesses and prevention of medical emergencies and dangerous behaviors.

In the following description, for the purposes of explanation, specific details are set forth in order to provide a thorough understanding of examples of the disclosure. However, it will be apparent that various examples may be practiced without these specific details. For example, devices, systems, structures, assemblies, methods, and other components may be shown as components in block diagram form in order not to obscure the examples in unnecessary detail. In other instances, well-known devices, processes, systems, structures, and techniques may be shown without necessary detail in order to avoid obscuring the examples. The figures and description are not intended to be restrictive. The terms and expressions that have been employed in this disclosure are used as terms of description and not of limitation, and there is no intention in the use of such terms and expressions of excluding any equivalents of the features shown and described or portions thereof. The word “example” is used herein to mean “serving as an example, instance, or illustration.” Any embodiment or design described herein as “example” is not necessarily to be construed as preferred or advantageous over other embodiments or designs.

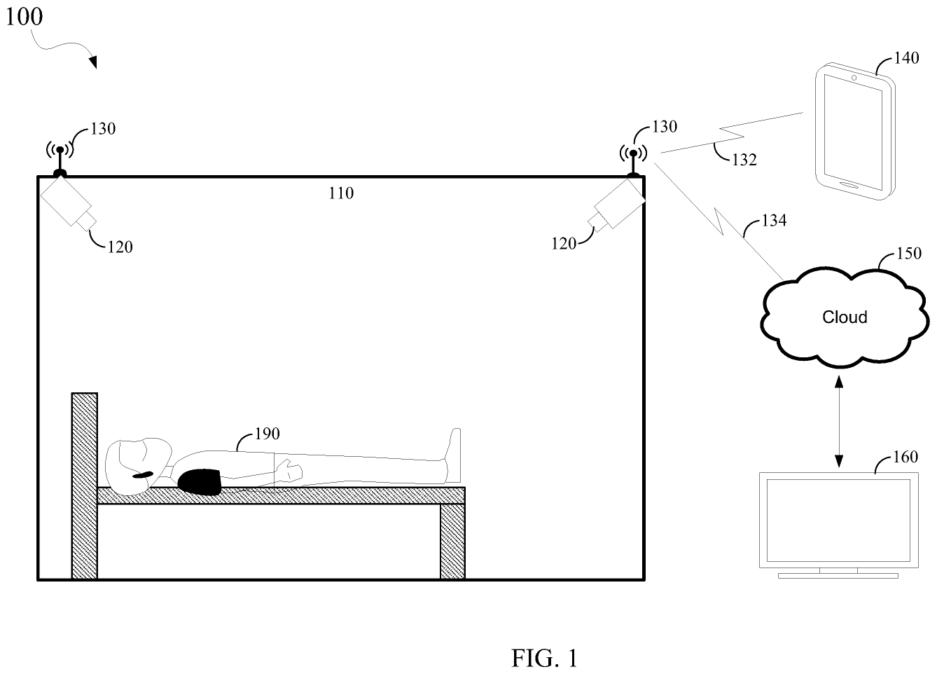

FIG. 1 illustrates an example of a system 100 for detecting abnormal behaviors indicative a certain illness based on movement event measurement according to certain embodiments. System 100 may include a structure 110 in which movement event measurement may be performed. Structure 110 may include, for example, a bedroom, a living room, or any other structure that may be frequented by a subject 190 to be monitored. In some embodiments, structure 110 may include a test lab or a patient room in a hospital. In the example shown in FIG. 1, structure 110 may be a bedroom where movement events of subject 190 at night may be monitored.

One or more motion sensors 120 may be set up in structure 110 to detect movement events of subject 190. For example, a motion sensor 120 may be set up at a location (e.g., installed on a wall or ceiling) in structure 110 and may be oriented such that a bed may be in the center of the field of view of motion sensor 120. Motion sensor 120 may include a remote, stationary motion sensor, rather than a wearable sensor or a sensor that is in physical contact with subject 190, such as a polysomnography or another sleep monitor that may be attached to the body of subject 190 to record various physiological signals. In some embodiments, motion sensor 120 may include a passive infrared motion sensor that can detect thermal energy emanated by subject 190 to detect motions of subject 190 based on changes in the detected thermal energy. In some embodiments, motion sensor 120 may include an active motion sensor that may include an infrared light (or acoustic or radio frequency signal) projector and an infrared light (or acoustic or radio frequency signal) detector.

Motion sensor 120 may generate movement data that includes a series of signals corresponding to a series of movement events of subject 190, which may also be referred to as coarse movement data. As used herein, a “movement event” may refer to an event in which a subject or a body part (e.g., an arm or a leg) of the subject moves into and/or out of a field of view of motion sensor 120. For example, a signal (e.g., a pulse, or a rising or falling edge of a pulse) may be generated by motion sensor 120 when subject 190 moves from a first location to a second location in structure 110. Some examples of motion sensors 120 and the coarse movement data are described in detail below.

A motion sensor 120 may include a wired or wireless communication subsystem for communicating with other electronic devices. For example, motion sensor 120 may send the series of signals associated with the detected movement events of subject 190 to a processing device through the wired or wireless communication subsystem, and/or may be controlled or configured through the wired or wireless communication subsystem. In the example shown in FIG. 1, motion sensor 120 may not include a wireless communication subsystem, but may send output signals to a data acquisition device 130, which may sample and collect the output signals of motion sensor 120 and then send the movement data that includes the series of signals to a computing system. For example, data acquisition device 130 may be based on a Raspberry Pi computer, and may send the collected movement data to a computing system 140 or to a cloud 150 through wired or wireless links 132 or 134. Computing device 140 may include, for example, a server, a mobile device, a tablet, a laptop computer, a desktop computer, or the like.

The movement data may be processed by computing system 140 or may be processed by another computing system 160 connected to cloud 150. For example, computing system 160 may be a cloud-based computing system that provides cloud computing services. Computing device 140 or 160 may process the movement data and detect, based on some reference data and/or a trained machine-learning model, an abnormal behavior that may be consistent with symptoms of a certain illness. Various techniques for processing the coarse movement data and detecting abnormal behaviors consistent with a certain illness are described in detail below.

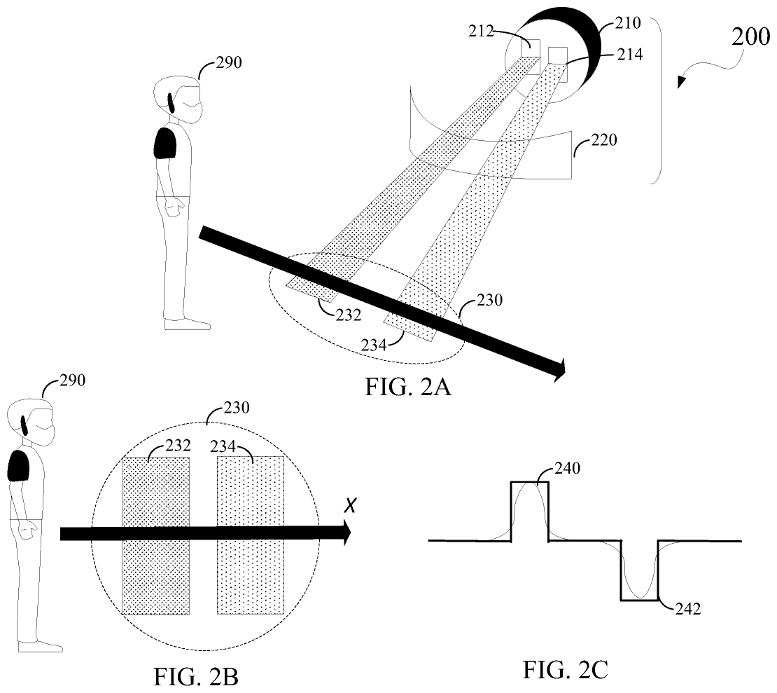

FIG. 2A illustrates an example of a motion sensor 200 for detecting movement events of a subject 290 according to certain embodiments. Motion sensor 200 may be an example of motion sensor 120. In the example shown in FIG. 2A, motion sensor 200 may include a passive infrared sensor that can detect whether a person has moved into or out of a field of view of the passive infrared sensor based on the measurement of infrared light emanating from the person in the field of view. At the normal body temperature, a person may radiate most strongly in the infrared band, such as infrared light with wavelengths around 10 μm. A passive infrared sensor may include one or more pyroelectric sensors (e.g., made of a ceramic material) that can generate surface charges when exposed to infrared radiation. For example, the pyroelectric sensors may generate a sufficiently high output voltage level even when the thermal signals are far below one microwatt.

In the illustrated example, motion sensor 200 may include multiple (e.g., two) pyroelectric sensors 212 and 214 housed in a hermetically sealed metal can 210. Pyroelectric sensors 212 and 214 may be formed on a ceramic or crystal substrate and may be configured as a differential pair to detect changes in the infrared radiation level. The differential pair configuration may compensate for offsets caused by environmental temperature changes and other output variations that may be common to the two pyroelectric sensors, and thus may provide a better sensitivity for detecting small changes in the spatial temperature pattern.

A lens 220 may be used to focus infrared light emitted from a person (e.g., subject 290) onto pyroelectric sensors 212 and 214 to increase the range and sensitivity of pyroelectric sensors 212 and 214. As described in detail below, lens 220 may include, for example, one or more Fresnel lenses. Lens 220 may also act as a protective cover for pyroelectric sensors 212 and 214. In the illustrated example, lens 220 may focus infrared light from a first region 232 of a field of view 230 onto pyroelectric sensor 212, and may also focus infrared light from a second region 234 of field of view 230 onto pyroelectric sensor 214. When subject 290 is not in field of view 230, pyroelectric sensors 212 and 214 may receive approximately the same low amount of infrared radiation, and thus the differential output signal from the two pyroelectric sensors may be close to zero. When subject 290 moves across field of view 230, the movement events may be detected by motion sensor 200.

FIG. 2B illustrates an example of motion detection using motion sensor 200 of FIG. 2A and based on heat source movements according to certain embodiments. FIG. 2C illustrates an example of motion sensor output signals indicative movement events of subject 290 as shown in FIG. 2B according to certain embodiments. In the illustrated example, subject 290 may enter field of view 230 from the left side in the x axis. When subject 290 is in first region 232, more infrared light radiated by subject 290 may be focused onto pyroelectric sensor 212 than onto pyroelectric sensor 214. Therefore, pyroelectric sensor 212 may detect an increased amount of infrared radiation, while pyroelectric sensor 214 may detect a lower amount of infrared radiation. As such, a first differential signal 240 between output signals of pyroelectric sensors 212 and 214 may be generated. First differential signal 240 may be a pulse, such as an impulse or a rectangular pulse as shown in FIG. 2C, which may indicate a movement event of subject 290 entering a field of view (e.g., field of view 230) of motion sensor 200.

When subject 290 reaches the center of field of view 230, infrared light radiated by subject 290 may be approximately equally focused onto pyroelectric sensor 212 and pyroelectric sensor 214. Therefore, pyroelectric sensor 212 and pyroelectric sensor 214 may detect approximately equal amount of infrared radiation. As such, the differential output signal from the two pyroelectric sensors may be close to zero.

When subject is in second region 234, more infrared light radiated by subject 290 may be focused onto pyroelectric sensor 214 than onto pyroelectric sensor 212. Therefore, pyroelectric sensor 214 may detect an increased amount of infrared radiation, while pyroelectric sensor 214 may detect a much lower amount of infrared radiation. As such, a second differential signal 242 between output signals of pyroelectric sensors 212 and 214 may be generated. Second differential signal 242 may be a pulse, such as an impulse or a rectangular pulse as shown in FIG. 2C, and may have an opposite phase (or polarity) compared with first differential signal 240. Second differential signal 242 may indicate a movement event of subject 290 leaving a field of view (e.g., field of view 230) of motion sensor 200.

When subject 290 moves out of field of view 230, no or a relatively small amount of infrared light radiated by subject 290 may be focused onto pyroelectric sensor 212 and pyroelectric sensor 214. Therefore, pyroelectric sensor 212 and pyroelectric sensor 214 may detect approximately equally low amount of infrared radiation. As such, the differential output signal from the two pyroelectric sensors may be close to zero. Based on the change of the differential signal from a pulse and to an opposite pulse, a movement event of subject 290 crossing field of view 230 may be detected.

As described above, lens 220 may be used to increase the range of the passive infrared sensors. Lens 220 may condense light from a large area onto a small area on the passive infrared sensors, and thus may change the breadth of the sensing region, provide a larger range of infrared energy to the passive infrared sensors, and improve the sensitivity of the passive infrared sensors. Additionally, lens 220 may be used to relatively easily change the sensing pattern. For example, lens 220 may include multiple Fresnel lenses molded on a thin plastic layer in the shape of a hemisphere, where the surface of the hemisphere may be divided into multiple facets, and each facet may include a Fresnel lens having a respective configuration for focusing light from a respective field of view onto the infrared sensitive sensors. Thus, the different Fresnel lenses may create multiple detection areas (e.g., different fields of view) corresponding to different solid angles in the three-dimensional space. To focus light from a respective field of view onto the infrared sensitive sensors, each Fresnel lens may have a different respective orientation and/or center offset. In some embodiments, every other Fresnel lens may point to a same pyroelectric sensor in a pair of pyroelectric sensors.

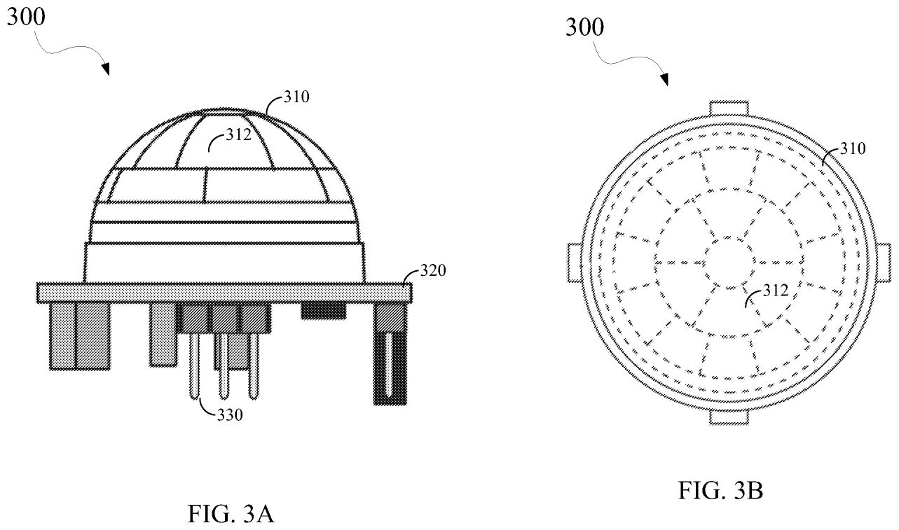

FIG. 3A is a side view of an example of a motion sensor 300 for detecting movement events of a subject according to certain embodiments. FIG. 3B is a top view of the example of motion sensor 300 for detecting movement events of a subject according to certain embodiments. Motion sensor 300 may include a lens 310 on top of a printed circuit board (PCB) 320 and at least partially covering PCB 320. Lens 310 may include multiple sub-lenses formed on multiple facets 312 of a substantially hemispherical structure. The substantially hemispherical structure may be made of a thin layer of plastic or glass material. The multiple facets may be configured based on the desired fields of view of motion sensor 300. Each sub-lens may include a Fresnel lens molded in the thin layer of plastic or glass material. The Fresnel lenses may have different orientations and center locations, and thus may focus infrared light from different fields of view onto the infrared sensors, such as pyroelectric sensors 212 and 214 described above. For example, each Fresnel lens may focus infrared light in a different range of solid angles onto the infrared sensors.

PCB 320 may include various electronic components installed thereon, such as the pyroelectric sensors formed on a ceramic or crystal substrate and housed in a metal can, power circuits, batteries, passive components such as capacitors and resistors, and the like. PCB 320 may also include connectors 330 installed thereon. Connectors 330 may be used to, for example, receive inputs or power from other devices and/or send the outputs of motion sensor 300 to other devices.

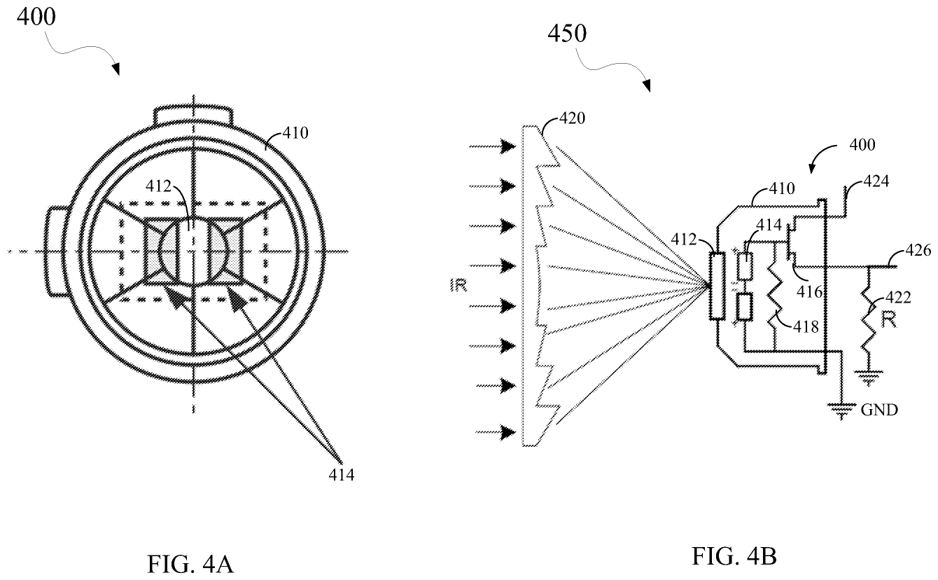

FIG. 4A illustrates an example of a passive infrared sensor 400 that includes two infrared sensors 414 for detecting movement events of a subject according to certain embodiments. As illustrated, passive infrared sensor 400 may include a metal can 410 and a window 412, where metal can 410 may be hermetically sealed. Infrared sensors 414 (e.g., pyroelectric sensors) may be housed by the hermetically sealed metal can 410 and may receive infrared light through window 412.

FIG. 4B illustrates a simplified block diagram of an example of a motion sensor 450 for detecting movement events of a subject according to certain embodiments. Motion sensor 450 may include passive infrared sensor 400 and a Fresnel lens 420. Fresnel lens 420 may focus infrared light onto passive infrared sensor 400 as described above. The infrared light may pass through window 412 and reach infrared sensors 414. Infrared sensors 414 may be sensitive to a wide range of radiation. To optimize for human detection, window 412 may include a filter that allows radiation in a range between about 8 μm and about 14 μm to pass through while blocking radiation outside of the range.

Infrared sensors 414 may include pyroelectric sensors made of ceramic materials that can generate surface charges when exposed to infrared radiation. The generated surface charges may increase as the amount of radiation changes. The charges may generate a voltage signal across a resistor 418 associated with infrared sensors 414. A field effect transistor (FET) 416 may be used to buffer this voltage signal. FET 416 may be a junction field effect transistor (JFET) with very low noise and may function as a source follower impedance converter. FET 416 may be connected to a voltage source (e.g., a 3.3-V or 5-V voltage source) through an input pin 424. An external resistor 422 connected to an output pin 426 may convert the FET current to an output voltage signal. The output voltage signal may be a function of the amount of infrared sensed by the pyroelectric sensors.

As shown in FIG. 4B, the two pyroelectric sensing elements in infrared sensors 414 may be connected in a way such that their outputs may have opposite polarities, and thus the voltage signal at the gate of FET 416 may be a function of the difference between the two outputs. As such, any signal common to both pyroelectric sensing elements may be canceled. Such an arrangement may cause a body passing in front of motion sensor 450 to activate one pyroelectric sensing element and then the other pyroelectric sensing element, while the vibration or other background signals that may affect both pyroelectric sensing elements simultaneously may be cancelled to reduce noises and improve sensitivity.

In some embodiments, a passive infrared sensor may include one pyroelectric sensing element or three or more pyroelectric sensing elements. For example, in one embodiment, a passive infrared sensor may include four pyroelectric sensing elements arranged at vertices of a square or a rectangle to cancel common signals in two dimensions.

In some embodiments, motion sensor 450 may also include a data collection system (not shown in FIG. 4B) implemented using, for example, a small computer, such as a Raspberry Pi. The Raspberry Pi is a small computer with a processor, peripheral connection slots, and general purpose input-output (GPIO) pins. Passive infrared sensor 400 may be connected to Raspberry Pi via input pin 424, output pin 426, and a ground (or reference) pin. For example, the ground pin on passive infrared sensor 400 may be connected to the ground on Raspberry Pi. Input pin 424 on passive infrared sensor 400 may be connected to the 5-V output pin on Raspberry Pi. Output pin 426 on passive infrared sensor 400 may be connected to a GPIO pin on Raspberry Pi for data acquisition.

The data acquisition system may sample the outputs at output pin 426 at a certain frequency, such as about 10 Hz (i.e., ten samples per second), 5 Hz, 2 Hz, 1 Hz, or the like. The sample frequency may be set based on the nature of the movement events to be detected. For example, a higher sampling frequency may be used for detecting motions that may include fast motions. In general, the higher the sampling frequency, the larger the collected movement data may be.

In some embodiments, the outputs at output pin 426 may be digitized by a digitizer in the data acquisition system. In some embodiments, the digitizer may be a comparator with one input pin electrically connected to output pin 426 and another input pin connected to a threshold voltage level, such that each sample may be converted to a one-bit value. For example, when the output at output pin 426 at a sampling time is higher than the threshold voltage level, a single-bit binary value “1” (or “0”) may be generated and saved. When the output at output pin 426 at a sampling time is lower than the threshold voltage level, a single-bit binary value “0” (or “1”) may be generated and saved. Thus, the movement data may include a series of single-bit values “1” and “0,” where a value “1” may indicate that a movement event occurs. Because the outputs may be sampled at a pre-set constant sampling frequency, the time stamps for the samples may be determined based on the sequence number of each sample (or bit) in the series of samples (or bits) in the collected movement data. Thus, the time stamps of the movement events (e.g., represented by “1s” in the movement data) may be determined based on the sequence numbers of the bits with values of “1” and the sampling frequency. In some embodiments, the outputs at output pin 426 may be sampled and digitized to n-bit values using an n-bit analog-to-digital converter.

In some embodiments, alternatively or additionally, the data acquisition system may record the time stamps of the detected movement events and save the time stamps in the movement data. Thus, the movement data may include the time stamps associated with detected movement events. In one example, the movement data may include a series of time stamps, rather than the sampled and digitized output values of the passive infrared sensor as described above. Each time stamp in the series of time stamps may indicate the time when a movement event occurs. In this way, the size of the movement data may be further reduced when the movement events are sparse, because the movement data would not include sequences of consecutive zeros indicating no detected movement events.

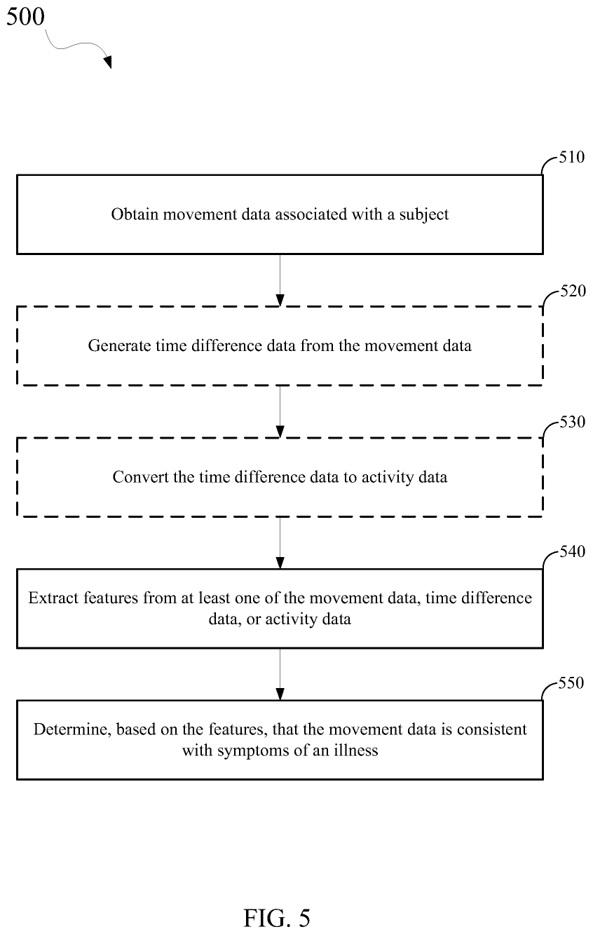

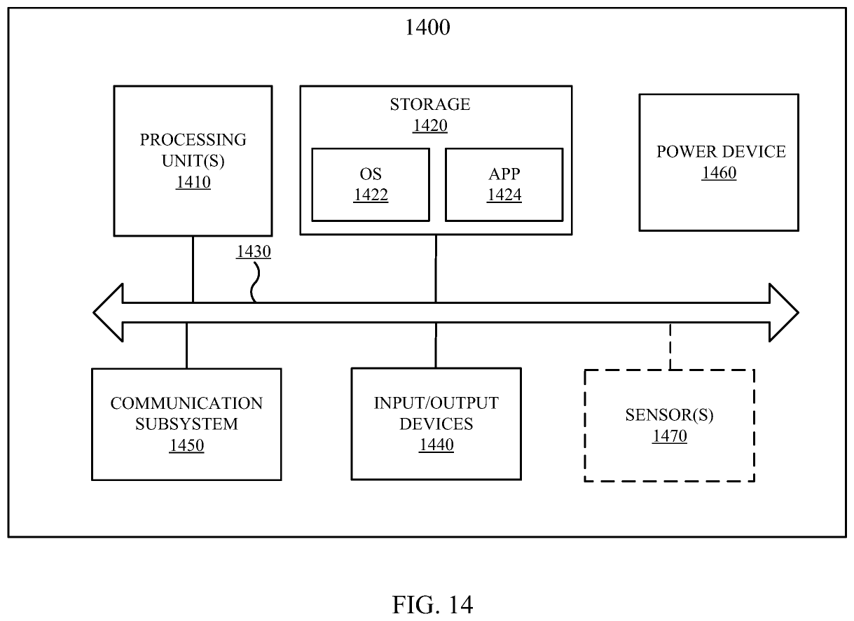

FIG. 5 includes a flow chart 500 illustrating an example of a method of detecting abnormal behaviors indicative a certain illness based on coarse movement data according to certain embodiments. Operations described in flow chart 500 may be performed by a computing system, such as computing system 140 or 160 described above with respect to FIG. 1 or a computing system described below with respect to FIG. 14. Although flow chart 500 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in flow chart 500 may begin at block 510, where a computing system may obtain movement data associated with a subject. The movement data may be collected by a motion sensor described above. For example, to diagnose obstructive sleep apnea, one or more motion sensors described above may be set up in a bedroom (e.g., installed on a wall or ceiling) to detect movement events of the subject during sleeping at night. The movement data generated by the one or more motion sensors may include a series of values representing a series of movement events of the subject (or a part of the subject's body) moving into and/or out of a field of view of a motion sensor, where the motion sensor may have one or more fields of view arranged according to a pattern as described above with respect to, for example, FIGS. 3A and 3B. In one example, the series of values may be a series of time stamps, where each time stamp corresponds to the time when the movement event is detected. In another example, the series of values may be ones and zeros, where the ones may indicate movement events, and the sequence or sample numbers of the ones may indicate the time instants when the movement events occur. In yet another example, the series of values may include a series of analog or digitized pulses. The movement data may be stored locally, on a remote server, or in the cloud. The computing system, which may include a mobile computer, a desktop computer, a server, or a cloud-based computing system, may obtain the movement data in real time as the movement data is collected, or may read stored movement data from a storage device, such as a hard drive, a server, or a cloud-based storage device.

Optionally, in some embodiments, the computing system may process the obtained movement data to remove some noise, errors, or other artifacts. For example, WiFi noise may be periodic interference noise that can cause the Raspberry Pi GPIO pin to record a digital high even though the passive infrared sensor outputs a digital low. The computing system may remove the periodic WiFi noise before further processing the movement data.

Optionally, at block 520, the computing system may generate time difference data based on the movement data. In some embodiments, time intervals between adjacent movement events in a series of movement events may be determined based on the time stamps in the movement data, for example, by subtracting the time stamp associated with a movement event from the time stamp associated with the next movement event. The time intervals between the adjacent movement events may be used as the time difference data. In some embodiments, other data, such as the integral or the derivative of the time difference data over time, may be directly or indirectly calculated using the time difference data and used for detecting abnormal behaviors.

In some embodiments, at block 530, the computing system may convert the time difference data into activity data. The activity data may indicate the instantaneous frequencies of the movement events at different time instants. For example, the activity data may be generated by calculating the inverse value of each time interval in the time difference data. In some embodiments, other data may be generated based on the movement data. For example, a total number of movement events detected during each respective time period (e.g., every 10 minutes, every half hour, every hour, etc.) may be counted and saved in a vector.

At block 540, the computing system may extract features from the movement data and/or other types of data generated based on the movement data, such as the time difference data, the activity data, or data including some other information generated from the movement data. More details of the feature extraction are described below. In some embodiments, the features may be scaled or normalized to remove certain effects (e.g., systematic variations) caused by different measurement settings for different sets of movement data.

At block 550, the computing system may determine whether the movement data is consistent with symptoms of a certain illness by feeding the extracted features to a machine-learning model, such as a classifier based on a logistic regression model, support vector machine, decision tree, nearest neighbor, Bayes classifier, boosted tree, hidden Markov model, neural network, or the like. Based on the determination results from the machine-learning model, the subject may be classified as likely or unlikely to be afflicted with the illness or have a certain abnormal behavior. In some embodiments, the machine-learning model may be used to predict future behaviors of the subject (e.g., violent behaviors or low performance) using the extracted features. The machine-learning model may be trained, for example, using features extracted from training movement data that includes movement data of subjects known to be afflicted with the illness and movement data of subjects known to be free of the illness in a supervised learning process. In some embodiments, a forward feature selection technique may be used to rank and select the most discriminative features for the classification. In some embodiments, the machine-learning model may also estimate a confidence level of the determination, a probability that the subject is afflicted with the illness, or an illness severity value. In some embodiments, the computing system may generate an output indicating the results of the determination, such as the classification results, the confidence level, and the like.

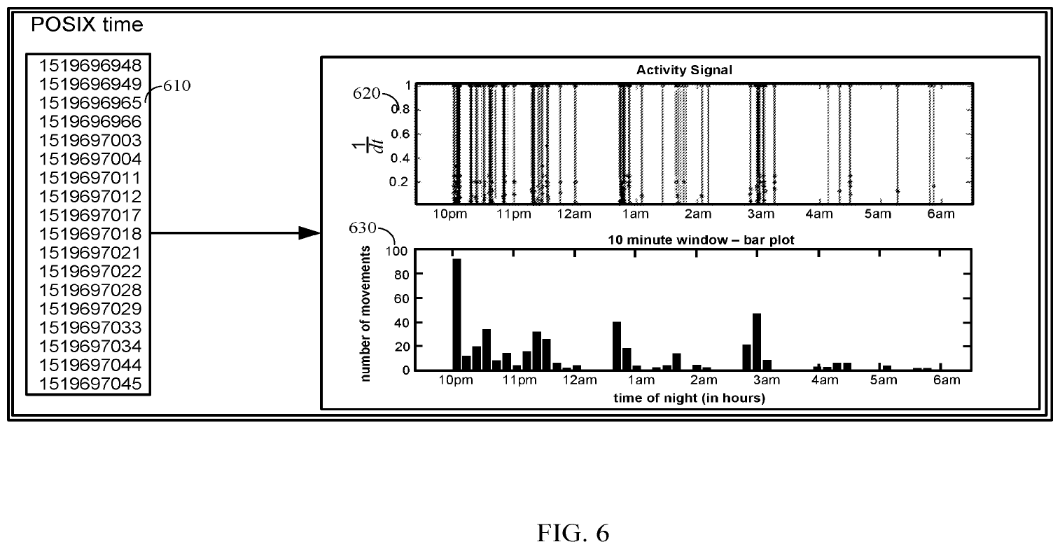

FIG. 6 illustrates an example of pre-processing measured movement data according to certain embodiments. As described above, the movement data may include a series of values representing a series of movement events of the subject (or a part of the subject's body) moving into and/or out of a field of view of the motion sensor. For example, the movement data may include a series of time stamps 610 in Portable Operating System Interface time (POSIX-time) format, where each time stamp may correspond to the time when the respective movement event is detected. In some embodiments, the movement data may include a series of impulses (or ones), and the associated time stamps for the impulses may be determined, for example, based on the sampling frequency and sample sequence numbers of the impulses, to generate the series of time stamps 610 in the POSIX-time format. In one example, the sampling frequency may be set to 1 Hz. Thus, a maximum number of impulses in the movement data of a subject acquired between 10 pm and 6 am the next day (about 8 hours) may be about 8×3600=28800 impulses. The time stamps of the impulses in the movement data may be concatenated to generate the series of time stamps 610 in the POSIX-time format as shown in FIG. 6.

FIG. 6 also shows that activity data 620 may be generated from the series of time stamps 610 in the POSIX-time format. For example, the time difference between two adjacent time stamps in the series of time stamps 610, which may indicate the time interval between two consecutive movement events, may be calculated to generate the time difference data. Thus, if a first movement event occurs at time t₁ and a second movement event occurs at time t₂, the time difference may be Δt=t₂−t₁. Activity data 620 may be generated by taking the inverse of each time difference value in the time difference data, which may indicate the instantaneous movement frequency at each movement event. Thus, each activity signal in activity data 620 may be calculated for a time instant when a respective movement event occurs by taking the inverse value of the time interval between the movement event and the previous movement event. For example, the activity signal for time t₂ may be 1/Δt. Thus, each activity signal in the activity data may inherently associate with the time of occurrence information relative to the previous activity signal. FIG. 6 shows activity data 620 plotted for each movement event. When the sampling frequency of the movement data is set to 1 Hz, the possible minimum time span between two movement events is one second, whereas the maximum time span may be the total monitoring time, such as 8 hours. Thus, the possible values in the activity data may be between 1 and about 0 as shown in FIG. 6. FIG. 6 also includes a bar plot 630 showing the number of movement events in each non-overlapping time window, such as in every 10 minutes, during the monitoring time period. The activity data plot and bar plot shown in FIG. 6 may help to visualize the activities of the subject during the monitoring time period.

In some embodiments, a user interface device (e.g., a touch screen) may be used by researchers or medical personnel to monitor and control (e.g., start or stop) the recording. For example, a graphic user interface (GUI) may provide real-time visualizations of the movement data, the time difference data, the activity data, and/or certain statistical data as shown in FIG. 6 to researchers or medical personnel. Based on the real-time data visualizations, researchers or medical personnel may, for example, change the sampling frequency of the data collection or take other appropriate actions.

In some embodiments, noise, errors, or other artifacts may be removed from the movement data before the time difference data and the activity data are generated. After the pre-processing of the movement data, various features may be extracted from the movement data and other types of data generated form the movement data, such as the time difference data and the activity data, and may be used to determine whether the behavior of the subject is consistent with the symptoms of a certain illness.

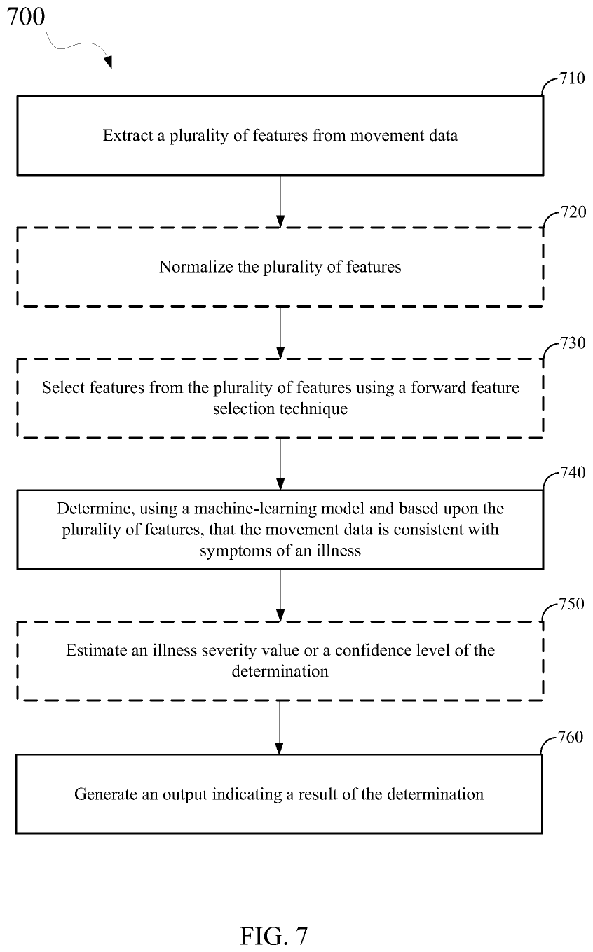

FIG. 7 is a flow chart 700 illustrating an example of a method of detecting abnormal behaviors indicative a certain illness based on pre-processed movement data according to certain embodiments. Operations described in flow chart 700 may be performed by a computing system, such as computing system 140 or 160 described above with respect to FIG. 1 or a computing system described below with respect to FIG. 14. Although flow chart 700 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

At block 710, a computing system may extract a plurality of features from movement data and other types of data generated form the movement data, such as time difference data and activity data. For example, the features may include statistical parameters of the activity data, such as a shape or a scale of the Generalized Pareto Distribution (GPD) of the activity data. Alternatively, or additionally, the features may include multiscale entropies for a plurality of different time scales, a mean of the multiscale entropies, or a variance of the multiscale entropies. In some embodiments, the features may also include statistical parameters of the time difference data, such as a mean, variance, skewness, kurtosis, or interquartile range of the time difference data.

GPD is a family of continuous probability distributions. GPD may be used to model the tails of another distribution. A GPD may be specified by three parameters including a location μ, a scale σ, and a shape ξ. A GPD may sometimes be specified only by the scale σ and shape ξ, or only by its shape ξ. The distribution function of GPD may be a conditional excess distribution function for estimating the distribution function of a variable x above a certain threshold μ, and may be defined as, for example:

F_{μ}(γ)=P(x−μ≤y|x>μ), 0≤y≤x_{F}−μ,

where x is a random variable, μ is a given threshold, y=x−μ is the excesses, and x_{F} is the right endpoint of the distribution function. A random variable x may have a generalized Pareto distribution if the cumulative distribution function (CDF) F(x) of variable x is given by:

$$F ( x ) = \{ \begin{matrix}1 - {( 1 + ξ z )}^{- 1 / ξ} & ξ \neq 0 \\ 1 - exp ( - z ) & ξ = 0\end{matrix} ,$$

or if a probability distribution function (PDF) f(x) of variable x is given by:

$$f ( x ) = \{ \begin{matrix}\frac{1}{σ} {( 1 + ξ z )}^{- 1 - 1 / ξ} & ξ \neq 0 \\ \frac{1}{σ} exp ( - z ) & ξ = 0\end{matrix} , where z = \frac{x - μ}{σ} .$$

Since the activity signals in the activity data have values greater than 0 as described above, μ may be 0 for all activity data. Thus, the GPD of the activity data may be characterized by the scale σ and shape ξ, which may be estimated from the activity data and used as the first set of features.

In some embodiments, entropy features may be extracted from the activity data. Traditional entropy analysis may analyze the regularity of a time series using a single time scale. However, the regularity of a time series may not adequately indicate the complexity of the time series. Multiscale entropy (MSE) analysis may be used to measure the complexity of a time series, such as the movement data or the activity data, by considering the complexity of the fluctuations inherent in the time series over a range of time scales. MSE may be used when the time scale of relevance in the time series is not known. The MSE analysis involves coarse graining or down sampling the time series, such that the time series is viewed at increasingly coarser time resolutions.

The MSE analysis may include two steps. The first step is the coarse graining process which may include several iterations. During each iteration, a coarse-grained time series Y^{(τ)} is built by averaging an original time series X including {x₁, x₂, . . . , x_{N}} over non-overlapping windows of length τ (also referred to as the scale factor or time scale).

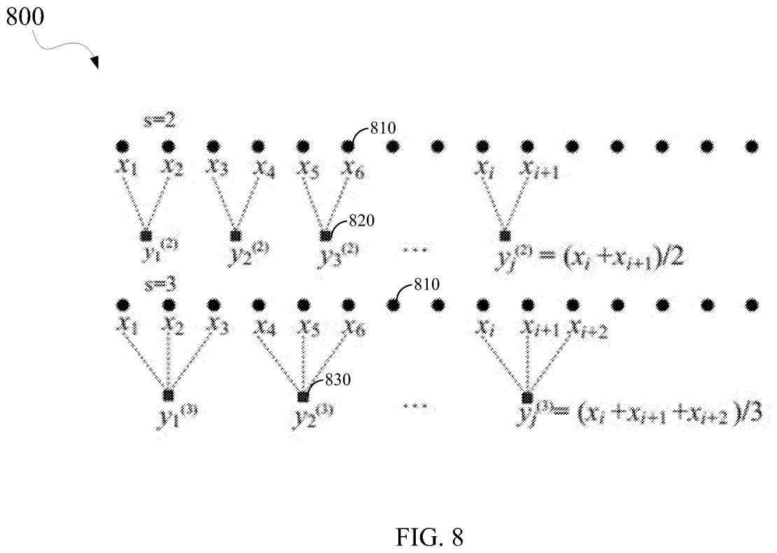

FIG. 8 illustrates an example 800 of generating multiscale time series Y^{(τ)} using a time series X according to certain embodiments. Time series X may include data points 810, such as x₁, x₂, x₃, . . . , and x_{N}, which may be sampled, for example, every second (and thus the original time scale τ is 1 second), where Nis the total number of data points 810 in time series X Coarse graining the data includes averaging different numbers of consecutive data points 810 to create different scales or resolutions of the signal. For example, at time scale τ=1, the coarse-grained time series Y⁽¹⁾ is the original time series X. At time scale τ=2, the coarse-grained time series Y⁽²⁾ may be formed by averaging two consecutive data points 810 to generate a data point 820 in time series Y⁽²⁾ as shown in FIG. 8. For example, y₁⁽²⁾=(x₁+x₂)/2, y₂⁽²⁾=(x₃+x₄)/2, and so on. At time scale τ=3, the coarse-grained time series Y⁽³⁾ may be formed by averaging three consecutive data points 810 to generate a data point 830 in time series Y⁽³⁾ as shown in FIG. 8. For example, y₁⁽³⁾=(x1+x2+x3)/3, y₂⁽³⁾=(x4+x5+x6)/3, and so on. The coarse-graining procedure may be performed one or more times according to:

$${y}_{j}^{( τ )} = \frac{1}{τ} {\sum }_{i = ( j - 1 ) τ + 1}^{j τ} {x}_{i} , 1 \leq j \leq N / τ .$$

In the second step, entropy is measured for each coarse-grained time series corresponding to time scale τ. Different measures of entropy, such as approximate entropy or sample entropy, can be used as a metric of entropy. Both approximate entropy and sample entropy can measure the degree of randomness or regularity of a time series. Sample entropy seeks matching patterns throughout a timer series to calculate its degree of regularity. Sample entropy may take two parameters: the pattern length m and the similarity criterion r which is the tolerance for accepting pattern matches. The similarity criterion r is a positive real value and may usually be chosen from a range between about 10% and about 20% of the standard deviation of the time series. Two patterns of length m match if every data point in the first pattern is within a distance r from the corresponding data point in the second pattern. The distance between two vectors may be defined as the maximum absolute difference between the components of the two vectors. For a vector

xₘ(i)={xᵢ. . . ,x₍ᵢ₊ₘ₎₋₁}, 1≤i≤N−m,

which is a pattern of length m in time series X, nᵢ^{m}(r) represents a total number of vectors xₘ(j) that have a distance smaller than r from xₘ(i), where i≠j to exclude the self matches. Uᵢ^{m}(r)=nᵢ^{m}(r)/(N−m) indicates the probability that the distance between vector xₘ(i) and any other vector xₘ(j) is smaller than r. U^{m}(r) is the probability of any two vectors of length m being within a distance r from each other. The probability U^{m}(r) may be determined by:

$${U}^{m} ( r ) = 1 / ( N - m ) {\sum }_{i = 1}^{N - m} {U}_{i}^{m} ( r ) .$$

Sample entropy may be determined according to:

$${\hat{H}}_{SE} ( m , r ) = {lim}_{N \to ∞} - ln \frac{{U}^{m + 1} ( r )}{{U}^{m} ( r )} ,$$

and may be estimated according to:

$${\hat{H}}_{SE} ( m , r , N ) = - ln \frac{{U}^{m + 1} ( r )}{{U}^{m} ( r )} .$$

Sample entropy may be less dependent on the time series length than the approximate entropy, and thus may be relatively consistent over a broader range of possible r, m, and N values.

Sample entropy may be computed for each of the time scales or resolutions and plotted vs the scale to generate a curve. The area under the curve, which may be the sum of sample entropy values over the range of scales, may be used as a multiscale entropy measure. A time series that has a lot of fluctuations may be associated with a higher entropy values and thus can be regarded as a signal with a higher complexity, while signals with high degrees of regularity may have lower entropy values.

In some embodiments, additional features may be extracted from the MSEs. For example, MSEs at multiple scales, such as {1, 2, 3, 4, 5, . . . , M}, may be determined as described above, and the mean and variance of the MMSE features may be calculated as additional features.

In some embodiments, some features may be derived from the time difference data. For example, the total number of impulses in the movement data (or the total number of samples in the time difference signal plus 1) may be used as a feature. The mean, variance, skewness, kurtosis, inter-quartile range, and the like, of the time difference data may be calculated and used as features for abnormal behavior detection and classification.

Referring back to FIG. 7, at block 720, the plurality of features may optionally be scaled or normalized to remove certain effects caused by, for example, differences in the structure (e.g., different rooms) and the positioning and configuration of the motion sensor in the structure. For example, when the movement data of different subjects or a same subject is collected in different settings, such as in different rooms, using different motion sensors, or using motion sensors having different arrangements in different rooms, movement data captured in each respective setting may be normalized by, for example, subtracting from each extracted feature the mean value of corresponding features extracted from movement data collected in the same respective setting.

Optionally, at block 730, the computing system may select features from the plurality of features (e.g., a total of K features) using, for example, a forward feature selection technique. For different abnormal behaviors or illnesses, the features that may be most discriminative for the detection or classification may be different. Thus, it may be desirable to rank and select the most discriminative features for a particular abnormal behavior or illness. A forward feature selection technique may be used to rank and select the most discriminative features for the classification, for example, during the training of a machine-learning model. During the inference using the trained machine-learning model, the most discriminative features may be extracted and used.

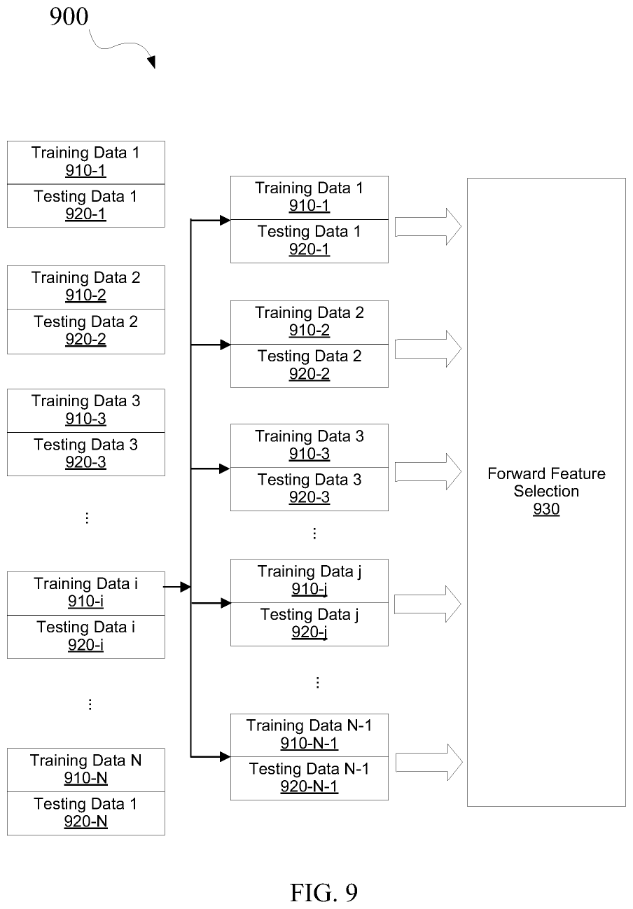

FIG. 9 illustrates an example of forward feature selection-based classification using a leave-one-out cross-validation method according to certain embodiments. In the example shown in FIG. 9, movement data associated with N subjects with known medical conditions (e.g., afflicted with or free of obstructive sleep apnea) is collected and used as training datasets 1-N (e.g., 910-1, . . . , 910-i, . . . , and 910-N) and/or testing datasets 1-N (e.g., 920-1, . . . , 920-i, . . . , and 920-N). The leave-one-out cross-validation analysis may be used to rank and select the most discriminative features from K features in N iterations. In each iteration, movement data corresponding to one subject in the N subjects may be used as the testing dataset to test K classifiers built using the training datasets associated with the remaining N−1 subjects. The kth classifier in the K classifiers may correspond to a model trained using only k features of the K features extracted from the movement data.

Based on the leave-one-out cross-validation analysis results, a forward feature selection engine 930 may rank and select the most discriminative features from the K features. For example, the accuracy, sensitivity, specificity, and average of sensitivity and specificity of the classifiers built using different numbers of features and different combinations of features may be calculated and used to select the most discriminative features from the K features. The corresponding model trained using these most discriminative features may be used for inferences.

Referring back to FIG. 7, at block 740, the computing system may determine, using a machine-learning model (e.g., a binary classifier) and based upon the plurality of features, that the movement data is consistent with symptoms of a certain illness. The machine-learning model may determine that the movement data associated with the subject under test is (or is not) consistent with symptoms of the illness by feeding the extracted features to a machine-learning model. The machine-learning model may include, for example, a classifier based on a logistic regression model, support vector machine, decision tree, nearest neighbor, Bayes classifier, boosted tree, hidden Markov model, neural network, or the like. In some embodiments, the machine-learning model may be used to predict future behaviors of the subject using the features extracted from measured movement data. The machine-learning model may be trained, for example, using features extracted from training data that includes movement data of subjects known to be afflicted with a certain illness and also movement data of subjects known to be free of the illness. For example, the machine-learning model trained using the most discriminative features, as described above with respect to block 730 and FIG. 9, may be used to classify the subject under test as likely or unlikely to be afflicted with the illness or have a certain abnormal behavior, based on the most discriminative features extracted from the movement data associated with the subject. In some embodiments, the machine-learning model may also be used to perform the feature extraction described above.

At block 750, the computing system may optionally estimate a confidence level of the determination, a probability that the subject is afflicted with the illness, or an illness severity value. For example, the probability estimates of the classifier may be used to generate an obstructive sleep apnea severity score according to Apnea-Hypopnea Index (AHI), which is an indicator of the severity of obstructive sleep apnea measured using polysomnography. The severity of sleep apnea based on the AHI may be grouped into three categories including mild (e.g., about 5-15 events/hour), moderate (e.g., about 15-30 events/hour), and severe (e.g., >30 events/hour).

At block 760, the computing system may generate an output indicating a result of the determination, such as the classification result, the confidence level, and the like. For example, if the classification result is positive, the computing system may transmit the result or generate an alarm or warning signal to another electronic device, such as an electronic device used by a caregiver, medical facility, monitoring service, or hospital. The result may be received by the other electronic device and may enable a user of the other device (e.g., a caregiver or physician) to determine whether admission to a hospital or further medical care is to be recommended to the subject. Caregivers and physicians may also proactively contact the user to recommend changes in a treatment plan. In some embodiments, the other electronic device may communicate to the computing system or the data acquisition system to adjust a frequency or scope of the monitoring and data collection based on the received result. In this way, the configuration of the motion sensors and the data acquisition systems may be updated to ensure that caregivers and physicians receive an appropriate level of granularity in subject monitoring and abnormal predication.

### Examples

As described above, techniques disclosed herein can be used to detect or predict abnormal behaviors of subject using a machine-learning model based on movement data collected by stationary coarse motion sensors. The abnormal behaviors may indicate that the subjects are likely to be afflicted with an illness, such as obstructive sleep apnea, Alzheimer's disease, depression, neurological conditions, stoke, pneumonia, dementia, chronic pulmonary disorders, muscular disease, and the like. The techniques disclosed herein may also be applied to predict the likelihood of negative future behaviors (e.g., violent behaviors or low performance). Several experiments have been conducted using techniques disclosed herein to diagnose obstructive sleep apnea or mental illnesses, or to predict negative future behaviors.

Passive infrared motion detectors as described above were used to monitor movement events of 32 elderly male participants (age∈[61 years, 73 years]) during overnight sleep in a sleep lab. Data acquisition systems were set up in two rooms in the sleep lab. Overnight movement events of the participants were continuously recorded using the low cost off-body passive infrared motion detectors described above for up to 8 hours. A Raspberry Pi was used to record the output of each passive infrared motion detector and to upload the recorded movement data to a Health Insurance Portability and Accountability Act (HIPAA)-compliant repository in a cloud service. The passive infrared motion detector is connected to the Raspberry Pi via the GPIO pins for data acquisition. The passive infrared motion detector has 3 pins including a 5 V power input (IN) pin, a signal output (OUT) pint, and a ground (G) pin. The G pin on the passive infrared motion detector was connected to the ground or a ground pin on the Raspberry Pi. The IN pin on the passive infrared motion detector was connected to the 5 V output pin on the Raspberry Pi. The OUT pin on the passive infrared motion detector was connected to a GPIO pin (e.g., pin 21) on the Raspberry Pi. GPIO pin 21 was chosen for convenience in building the system. Other GPIO pins may be used in other configurations. Python and shell-based scripts were used to collect data and archive the data in the HIPAA-compliant repository. A touchscreen was used to control the recording. A graphical user interface (GUI) written in python was created for the control, which also provided real-time data visualizations.

Polysomnogram results were simultaneously measured and recorded for the participants, where breathing was measured with separate channels for oral/nasal airflow, nasal pressure, thoracic and abdominal respiratory effort, pulse oximetry, and the like. From the polysomnogram results, the participants were assigned different obstructive sleep apnea labels by an expert. Participants with an AHI value greater than or equal to 15 events per hour were assigned to the obstructive sleep apnea class. Participants with an AHI value less than 15 served as the control group. Among the cohort of 32 elderly male participants, 14 participants had severe obstructive sleep apnea and 18 participants were controls.

Overnight movement data Ri for ith participant Pi during a single night's recording includes Li impulse signals, where i is between 1 and N, and N (e.g., 32 in the study) is the number of participants in the data cohort. The sampling frequency (Fs) was set to 1 Hz. Thus, for the ith movement data Ri acquired between 10 pm in the night and 6 am the next morning (about 8 hours), there were at most 8×3600=28800 impulses, and thus Li≤28800. Time stamps in the POSIX-time format corresponding to impulses present in movement data Ri were concatenated to construct ith time series Ti in POSIX-time format. The ith time difference data Di was calculated based on the time differences between consecutive impulses in movement data Ri or between consecutive time stamps in time series Ti, and the ith activity data Ai was calculated based on sample-wise inverses of the ith time difference data Di. Because the sampling frequency was set to 1 Hz, the possible minimum time span between two movement events was 1 second, whereas the maximum time span could be as long as the data collection time. Thus, the activity signals in activity data Ai are bounded between 0 and 1, i.e., Ai[t]∈[0, 1]∀t∈[10 pm, 6 am].

For each participant Pi, 15 features were extracted from activity data Ai and time difference data Di generated from movement data Ri. As shown in Table 1 below, these 15 features belong to three groups, including the distribution, entropy, and time difference data statistics. The first set of features includes GPD parameters including the shape ξ and scale σ as described above. The next set of features is based on MSEs as described above. In the example shown in Table 1, MSE features at 5 time scales τ∈{1, 2, 3, 4, 5} were calculated, and then the mean and variance of these 5 MSE features were computed to obtain a total of 7 entropy based features. The third set of features is derived from the time difference data Di, rather than the activity data Ai. The third set of features includes the number of impulses in movement data Ri (or the number of samples in the time difference data Di plus 1), and the mean, variance, skewness, kurtosis, and inter-quartile range of the time difference data Di.

TABLE 1
Examples of Features Extracted from Time
Difference Data and Activity Data

| Feature type | Feature |
|---|---|
| Distribution | GPD shape parameter (ξ) |
| | GPD scale parameter (σ) |
| Entropy | MSE - Scale 1 (MSE₁) |
| | MSE - Scale 2 (MSE₂) |
| | MSE - Scale 3 (MSE₃) |
| | MSE - Scale 4 (MSE₄) |
| | MSE - Scale 5 (MSE₅) |
| | Mean of 5-scale MSE (μ_{MSE}) |
| | Variance of 5-scale MSE (σ_{MSE}²) |
| Time Difference Data | Number of impulses in the recording (L) |
| statistics | Mean of time difference data (μ_{d}) |
| | Variance of time difference data (σ_{d}²) |
| | Skewness of difference data (γ_{d}) |
| | Kurtosis of time difference data (K_{d}) |
| | IQR of time difference data (IQR_{d}) |

Two types of feature scaling were performed before the machine-learning classifier was built. First, to reduce or eliminate the effects of possibly different positions of the sensors in the two rooms in the sleep lab on the extracted features, room specific mean subtraction for each of the distribution and entropy-based features was performed as described above with respect to, for example, block 720. During training, feature specific z-scoring on the training data was performed and the extracted sample mean and sample variance were used to normalize the test data.

Based on the extracted features shown in Table 1, a logistic regression model was built to classify each participant into one of two classes (e.g., obstructive sleep apnea and control). A forward feature selection algorithm was used to rank and select the most discriminative features for the classification. Leave-one-out cross-validation analysis as described above with respect to FIG. 9 was performed using movement data for N=32 participants in 32 iterations. In each iteration of the cross-validation, data corresponding to one participant was used as the testing dataset, and 15 classifiers were built using the remaining datasets (corresponding to the remaining 31 participants). The kth classifier of the 15 classifiers corresponded to a model trained to classify obstructive sleep apnea versus control using k of the 15 features, where k∈[1, 15].

The list of experiments performed and the corresponding performance of the obstructive sleep apnea classification models are shown in Table 2. The different experiments shown in Table 2 correspond to different numbers of features used in the obstructive sleep apnea classification. The performance parameters of the obstructive sleep apnea classification models include accuracy, sensitivity, specificity, and an average of the sensitivity and specificity. The accuracy describes the ability of the model to correctly differentiate participants with obstructive sleep apnea and controls, and can be calculated according to:

accuracy=(TP+TN)/(TP+TN+FP+FN),

where TP (true positive) is the number of participants correctly identified as afflicted with obstructive sleep apnea, TN (true negative) is the number of participants correctly identified as controls (not afflicted with obstructive sleep apnea), FP (false positive) is the number of participants incorrectly identified as afflicted with obstructive sleep apnea, and FN (false negative) is the number of participants incorrectly identified as controls. As shown in Table 2 below, the best accuracy (e.g., about 81%) was achieved using 3, 5, 6, or 8 features.

The sensitivity describes the ability of the model to correctly determine the participants afflicted with obstructive sleep apnea, and can be calculated according to sensitivity=TP/(TP+FN). The specificity describes the ability of the model to correctly determine the participants not afflicted with obstructive sleep apnea, and can be calculated according to specificity=TN/(TN+FP). The sensitivity and specificity for all 15 experiments were calculated to better understand the classification performance. The mean of the sensitivity and the corresponding specificity is denoted as S_{μ}∈[0, 1]. In general, the higher the value of S_{μ}, the better the classification performance of the model. The best S_{μ} (e.g., 0.82) was achieved when 6 features were used to perform the classification. The top 6 features that were picked with the highest probability than other features by the training procedure in a decreasing order were MSE₅, σ_{d}², MSE₂, MSE₃, K_{d}, and L.

TABLE 2
Examples of Performance of the Logistic Regression Classifiers

| # of features | Accuracy (%) | Sn | Sp | S_{μ} = (Sₙ + Sₚ)/2 |
|---|---|---|---|---|
| 1 | 75 | 0.57 | 0.89 | 0.73 |
| 2 | 78 | 0.79 | 0.78 | 0.78 |
| 3 | 81 | 0.79 | 0.83 | 0.81 |
| 4 | 75 | 0.71 | 0.78 | 0.75 |
| 5 | 81 | 0.79 | 0.83 | 0.81 |
| 6 | 81 | 0.86 | 0.78 | 0.82 |
| 7 | 69 | 0.71 | 0.67 | 0.69 |
| 8 | 81 | 0.71 | 0.89 | 0.80 |
| 9 | 78 | 0.79 | 0.78 | 0.78 |
| 10 | 66 | 0.71 | 0.61 | 0.66 |
| 11 | 72 | 0.64 | 0.78 | 0.71 |
| 12 | 66 | 0.64 | 0.67 | 0.65 |
| 13 | 63 | 0.57 | 0.67 | 0.61 |
| 14 | 66 | 0.64 | 0.67 | 0.65 |
| 15 | 59 | 0.57 | 0.61 | 0.59 |

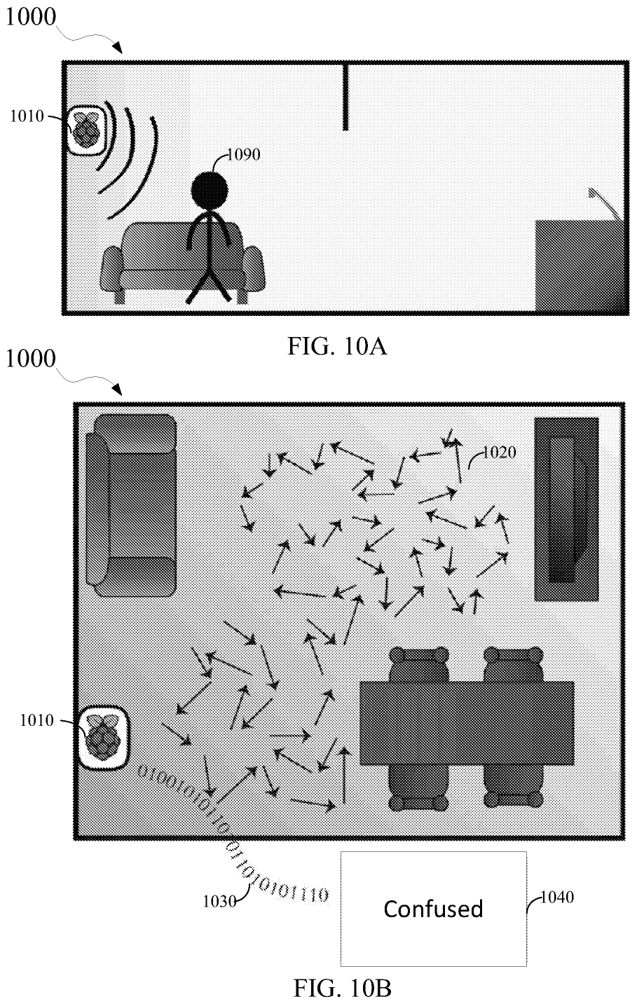

FIG. 10A is a side view of an example of a system 1000 for detecting abnormal behaviors indicative a certain mental illness based on movement event measurement according to certain embodiments. FIG. 10B is a top view of the example of system 1000 of FIG. 10A for detecting abnormal behaviors indicative the mental illness based on movement event measurement according to certain embodiments. System 1000 may be used to detect, for example, the way a subject moves around his/her home (or another) environment, how the subject interacts with others, and how the subject interacts with automated devices, to identify potential mental illnesses.

System 1000 may include a motion detector 1010 set up in a living room. Motion detector 1010 may include a motion sensor (e.g., passive infrared sensor) and a data acquisition system (e.g., implemented using Raspberry Pi) as described above. Motion detector 1010 may detect movement events 1020 of a subject 1090 in the living room, and record and transfer movement data 1030 associated with the detect movement events 1020. Movement data 1030 may include a series of binary values indicating whether movement events are detected at a series of time instants as described above. Movement data 1030 may be processed by a computing system 1040 to determine whether subject 1090 is likely to have a mental illness. For example, computing system 1040 may extract certain features from movement data 1030 and feed the extracted features to a machine-learning model that may classify subject 1090 as likely being confused or depressed. Computing device 1040 may generate an output of the determination result, such as sending a message or an alarm signal. For example, computing system 1040 may send an instruction to a device (e.g., a speaker) in the living room, and the device may play an audio message instructing subject 1090 to sit down and relax.

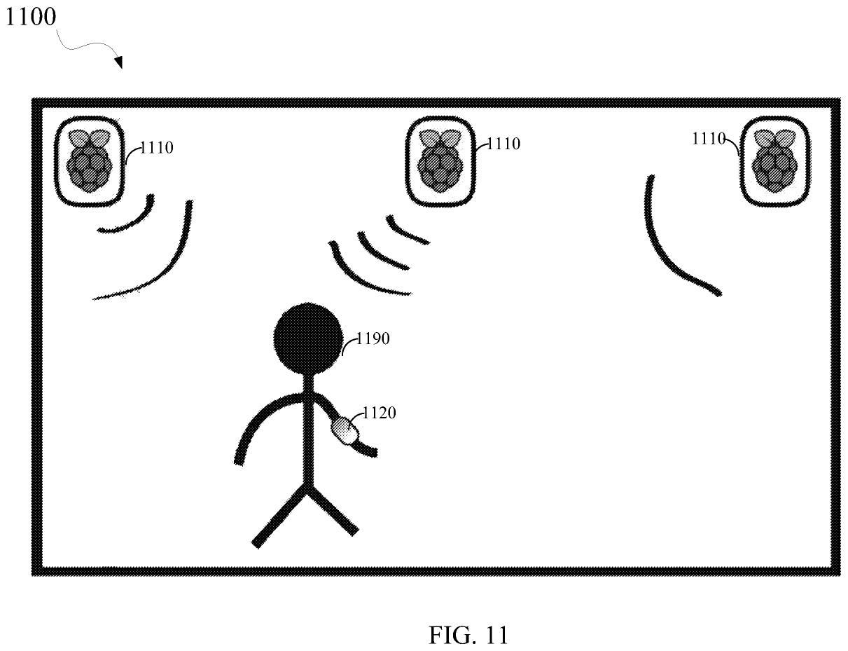

FIG. 11 illustrates another example of a system 1100 for detecting abnormal behaviors indicative certain mental illnesses or a certain negative future behaviors based on movement event measurement according to certain embodiments. System 1100 may be similar to system 1000, and may include two or more stationary motion detectors 1110 set up in a structure, such as a living room or a bedroom. In some embodiments, a subject 1190 being monitored may wear a wearable device 1120, which may help to improve the accuracy of movement sensing, person identification, and area localization, for example, when multiple subjects are present in the same room or structure. Wearable device 1120 may include, for example, a Bluetooth button on a pendant, a mobile phone, a smart watch, or another wearable device.

Stationary motion detectors 1110 and/or wearable device 1120 may determine locations or motions of subject 1190 by either passive sensing (e.g., passive infrared sensing devices placed in the room), or using WiFi, Bluetooth, or other radio frequency (RF) signals being emitted from subject 1190 (e.g., from wearable device 1120) or bouncing off subject 1190. The locations of subject 1190 may be determined by, for example, triangulation or trilateration. In some embodiments, the signal strength can be measured in two or three dimensions to infer the distances of subject 1190 to two or more sensors (e.g., stationary motion detectors 1110) set up at known locations, and the location of subject 1190 may then be determined to be at the intersect point of two or more circles or spheres with radiuses equal to the distances and with centers at the known locations of the two or more sensors. The movement events may then be determined based on the changes in the location of subject 1190. In some embodiments, movement events may be determined based on changes in the measured field or signal strength. The motion detectors may acquire data regarding subject 1190's patterns of movement as shown in, for example, FIG. 10B. The movement pattern may be indicative of an irregular, confused behavior or a normal behavior. The movement data may also be used to identify changes in the severity of a condition. For example, more random behaviors may indicate increasing in confusion, while more sedentary behaviors may indicate lethargy or increased symptoms of cardiovascular or respiratory diseases.

Processing the movement data using techniques disclosed herein can provide information on the severity of symptoms of a particular illness and may allow prediction of the severity for the next day. For example, using the time series of movement events and activities in spatially sensed areas, and with (or without) additional information such as temperature, time of day, season of year, other people present, or the like, a model may be built to predict if the behavior or symptoms for subject 1190 may deteriorate or improve in the near term, such that relatives or caregivers can provide appropriate interventions or reach out for help in a timely manner.

In some embodiments, a device in system 1000 or system 1100 may interact with the subject to suggest helpful next steps based upon, for example, the local context, such as the time of day, the location in building, and the history of activity at the same or similar time and place. For example, if the system determines that the subject may be in a confused state, it may be helpful to provide guided interactions with the subject to aid the subject self-correct any confused state that the subject may be experiencing. This may be accomplished by, for example, a vocal assistant interface that audibly interacts with the subject. The vocal assistant interface may suggest, for example, the completion of a current task, or the changing of tasks to something less stressful.

In some embodiments, a central unit, such as computing system 1040, may communicate via the internet or other networks (such as the phone system) to transmit alerts to family, friends, caregivers, or the like. For example, if the vocal assistant interface fails to aid the individual in self-correction during a confused state, the central unit may communicate with family members, caregivers, or others via a telecommunication system, such as through the internet, an active speaker, or a phone system.

In some embodiments, techniques disclosed herein may be used to predict feature behaviors of a subject, such as violent behaviors or lower performance. For example, a passive motion sensor as described above may be placed in a bedroom to record overnight movement events of a subject for predicting the behavior of the subject in the next day or a longer term. In one study, a data acquisition system including a passive infrared sensor and a Raspberry Pi was placed in the far field in a room to collect overnight movement data of a subject with behavioral issues over a period of two months. Each day, the recording was performed from 7 pm in the evening to 9 am the next morning. The Raspberry Pi recorded the time stamps when the passive infrared sensor detected a movement event. The behavior of the subject was logged daily. The behavior may be labeled as “good days” or “bad days,” where “bad days” may represent times when the subject would need extra attention, removal from general population, medication, or some other intervention to lessen the impact of the bad day on both the subject, any staff, and other residents in the facility.

Features may be extracted from the movement data as described above. For example, from the movement data, time difference data and activity data described above may be generated. Various features may then be extracted from the movement data, the time difference data, and/or the activity data as described above. The features may include, for example, GPD parameters including the shape ξ and scale σ as described above, MSE features at multiple scales and the mean and variance of the MSE features, the number of impulses in the movement data, and the mean, variance, skewness, kurtosis, and inter-quartile range of the time difference data.

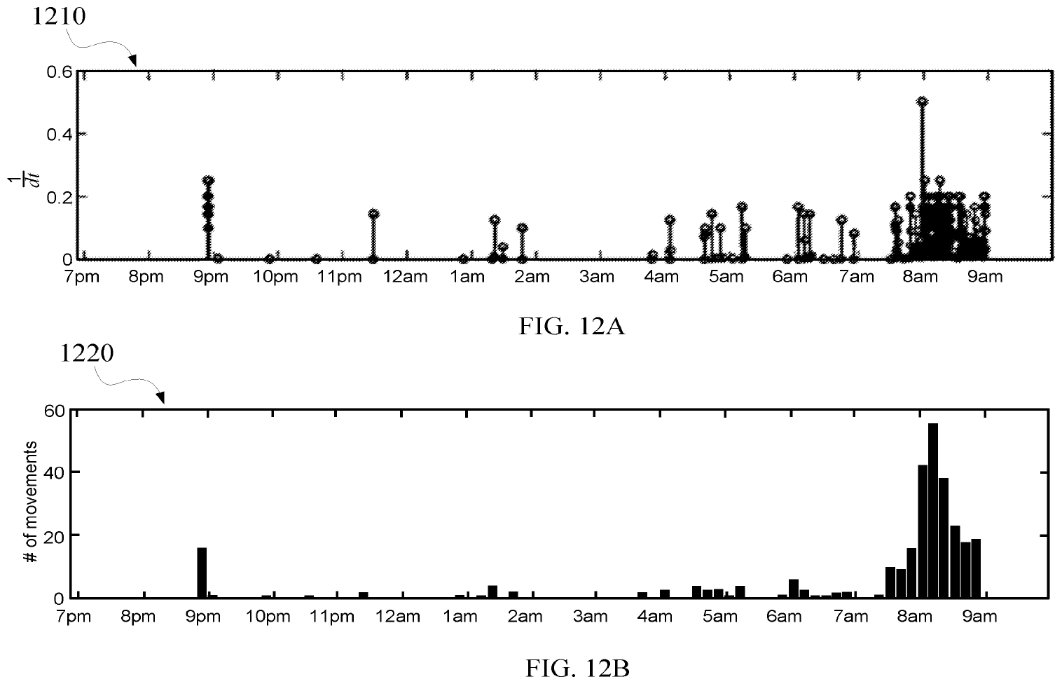

FIG. 12A illustrates an example of activity data 1210 including inverse values of time intervals between detected movement events of a subject according to certain embodiments. Activity data 1210 may be generated from movement data collected at a night from about 7 pm to about 9 am the next morning as described above. Each activity signal in activity data 1210 may be calculated for a time instant when a movement event occurred by taking the inverse value of the time interval between the movement event and the previous movement event. The sampling frequency of movement data was set to 1 Hz, and thus the possible minimum time span between two movement events was one second, whereas the maximum time span may be the total monitoring time, such as 14 hours (e.g., from about 7 pm to about 9 am the next morning). Thus, the values in the activity data may be between about 1 and about 0 as shown in FIG. 12A.

FIG. 12B illustrates an example of a bar plot 1220 showing numbers of movement events of the subject in different time windows according to certain embodiments. Bar plot 1220 may be generated from movement data collected at a night from about 7 pm to about 9 am the next morning as described above. In the example shown in FIG. 12B, each time window is about 10 minutes, and the monitoring time period is 14 hours (e.g., 7 pm-9 am). The plots shown in FIGS. 12A and 12B may help to visualize the activity of the subject through the night.

The following analysis was performed to predict a negative behavior during day time. GPD parameters, such as the scale σ and shape ξ were estimated from movement data collected at a single night to obtain a 2-dimensional GPD feature vector per night. Features were also extracted from the activity data generated from the movement data collected at the single night. MSE features for 10 time scales and the 2-dimensional GPD features together formed a 12-dimensional feature vector for each night. Classification was performed using features extracted from movement data collected in previous 1, 2, 3, or 4 nights, and the results are [17 Bad days, 11 Good days], [15 Bad days, 10 Good days], [13 Bad days, 9 Good days] and [12 Bad days, 7 Good days], respectively.

A least absolute shrinkage and selection operator (LASSO) or elastic net regularization for generalized linear model method was used for the classification. The LASSO parameter alpha was varied from 0.1 to 1 at a step 0.1, and the results were presented for each alpha value. For LASSO parameter lambda, 100 values were chosen from a geometric sequence ranging from 0.0001 to 3.5. The best lambda value was chosen by performing a cross-validation within the training data set. The data set was balanced using random sampling to obtain N data points, and a (N/2)-fold cross-validation was performed. The experiment was repeated 100 times. The accuracies were averaged over the different cross-validations and 100 iterations.

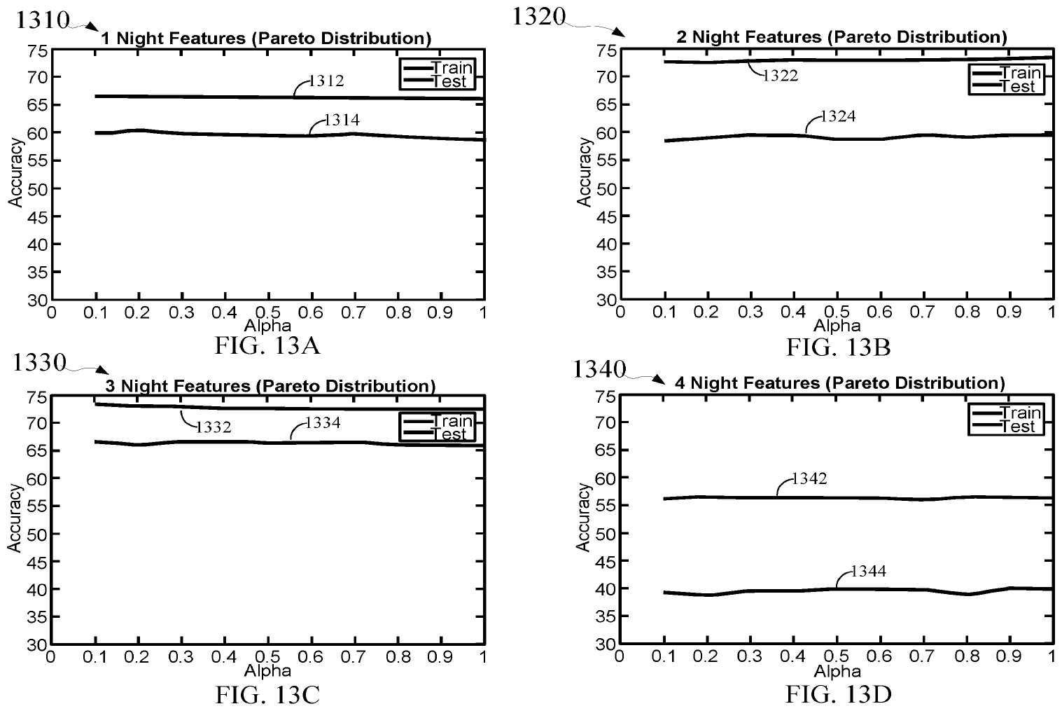

FIG. 13A illustrates examples of training and test accuracies using Generalized Pareto Distribution (GPD) features extracted from movement data for one night according to certain embodiments. The training accuracies at different alpha values are shown by a curve 1312, which shows a maximum training accuracy about 66%. The test accuracies at different alpha values are shown by a curve 1314, which shows a maximum test accuracy about 60%.

FIG. 13B illustrates examples of training and test accuracies using GPD features extracted from movement data for two nights according to certain embodiments. The training accuracies at different alpha values are shown by a curve 1322, which shows a maximum training accuracy about 74% The test accuracies at different alpha values are shown by a curve 1324, which shows a maximum test accuracy about 60%.

FIG. 13C illustrates examples of training and test accuracies using GPD features extracted from movement data for three nights according to certain embodiments. The training accuracies at different alpha values are shown by a curve 1332, which shows a maximum training accuracy about 74%. The test accuracies at different alpha values are shown by a curve 1334, which shows a maximum test accuracy about 66%.

FIG. 13D illustrates examples of training and test accuracies using GPD features extracted from movement data for four nights according to certain embodiments. The training accuracies at different alpha values are shown by a curve 1342, which shows a maximum training accuracy about 56%. The test accuracies at different alpha values are shown by a curve 1344, which shows a maximum test accuracy about 40%.

The results shown by FIGS. 13A-13D indicate that, for the collected data sets, using GPD features alone may give best test results. The highest test accuracy (e.g., about 66%) was achieved by using movement data for 3 nights and using GPD parameters as features, which implies that poor sleep may have a cumulative effect and may lead to behavioral changes that need intervention, and thus early intervention (e.g., sleep therapy) may lead to reduced incidents.

FIG. 14 is a simplified block diagram of an example of a computing system 1400 for implementing certain embodiments disclosed herein. Computing system 1400 may be used in the data acquisition system described above for collecting movement data, or may be used as the computing system for extracting features and applying machine-learning models to the extracted features for classification or prediction. The block diagram illustrates some electronic components or subsystems of the computing system. Computing device 1400 depicted in FIG. 14 is merely an example and is not intended to unduly limit the scope of inventive embodiments recited in the claims. One of ordinary skill in the art would recognize many possible variations, alternatives, and modifications. For example, in some implementations, Computing device 1400 may have more or fewer subsystems than those shown in FIG. 14, may combine two or more subsystems, or may have a different configuration or arrangement of subsystems.

In the example shown in FIG. 14, computing system 1400 may include one or more processing units 1410 and storage 1420. Processing units 1410 may be configured to execute instructions for performing various operations, and can include, for example, a micro-controller, a general-purpose processor, or a microprocessor suitable for implementation within a portable electronic device, such as a Raspberry Pi. Processing units 1410 may be communicatively coupled with a plurality of components within computing system 1400. For example, processing units 1410 may communicate with other components across a bus 1430. Bus 1430 may be any subsystem adapted to transfer data within computing system 1400. Bus 1430 may include a plurality of computer buses and additional circuitry to transfer data.

Storage 1420 may be coupled to processing units 1410. In some embodiments, storage 1420 may offer both short-term and long-term storage and may be divided into several units. Storage 1420 may be volatile, such as static random access memory (SRAM) and/or dynamic random access memory (DRAM), and/or non-volatile, such as read-only memory (ROM), flash memory, and the like. Furthermore, storage 1420 may include removable storage devices, such as secure digital (SD) cards. Storage 1420 may provide storage of computer readable instructions, data structures, program modules, audio recordings, image files, video recordings, and other data for computing system 1400. In some embodiments, storage 1420 may be distributed into different hardware modules. A set of instructions and/or code might be stored on storage 1420. The instructions might take the form of executable code that may be executable by computing system 1400, and/or might take the form of source and/or installable code, which, upon compilation and/or installation on computing system 1400 (e.g., using any of a variety of generally available compilers, installation programs, compression/decompression utilities, and the like), may take the form of executable code.

In some embodiments, storage 1420 may store a plurality of application modules 1424, which may include any number of applications, such as applications for controlling input/output (I/O) devices 1440 (e.g., a sensor, a switch, a camera, a microphone or audio recorder, a speaker, a media player, a display device, etc.). Application modules 1424 may include particular instructions to be executed by processing units 1410. In some embodiments, certain applications or parts of application modules 1424 may be executable by other hardware modules, such as a communication subsystem 1450. In certain embodiments, storage 1420 may additionally include secure memory, which may include additional security controls to prevent copying or other unauthorized access to secure information.

In some embodiments, storage 1420 may include an operating system 1422 loaded therein, such as an Android operating system or any other operating system suitable for mobile devices or portable devices. Operating system 1422 may be operable to initiate the execution of the instructions provided by the application modules 1424 and/or manage other hardware modules as well as interfaces with communication subsystem 1450 which may include one or more wireless or wired transceivers. Operating system 1422 may be adapted to perform other operations across the components of computing system 1400 including threading, resource management, data storage control, and other similar functionality.

Communication subsystem 1450 may include, for example, an infrared communication device, a wireless communication device and/or chipset (such as a Bluetooth® device, an IEEE 802.11 (Wi-Fi) device, a WiMax device, cellular communication facilities, and the like), NFC, ZigBee, and/or similar communication interfaces. Computing device 1400 may include one or more antennas (not shown in FIG. 14) for wireless communication as part of communication subsystem 1450 or as a separate component coupled to any portion of the system. Depending on desired functionality, communication subsystem 1450 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 802.9) network. A WLAN may be, for example, an IEEE 802.11x network. A WPAN may be, for example, a Bluetooth network, an IEEE 802.15x, or some other types of network. The techniques described herein may also be used for any combination of WWAN, WLAN, and/or WPAN. In some embodiments, communications subsystem 1450 may include wired communication devices, such as Universal Serial Bus (USB) devices, Universal Asynchronous Receiver/Transmitter (UART) devices, Ethernet devices, and the like. Communications subsystem 1450 may permit data to be exchanged with a network, other computing systems, and/or any other devices described herein. Communication subsystem 1450 may include a means for transmitting or receiving data, such as identifiers of portable goal-tracking devices, position data, a geographic map, a heat map, photos, or videos, using antennas and wireless links. Communication subsystem 1450, processing units 1410, and storage 1420 may together comprise at least a part of one or more of a means for performing some functions disclosed herein.

Computing device 1400 may include one or more I/O devices 1440, such as a sensor, a switch, a camera, a microphone or audio recorder, a communication port, or the like. For example, I/O devices 1440 may include one or more touch sensors or button sensors associated with the buttons. The touch sensors or button sensors may include, for example, a mechanical switch or a capacitive sensor that can sense the touching or pressing of a button. In some embodiments, I/O devices 1440 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP3, WAV, WMA, DSS, etc. The recorded audio files may be saved to storage 1420 or may be sent to the one or more network servers through communication subsystem 1450.

In some embodiments, I/O devices 1440 may include a camera, such as a high-definition pinhole camera or a camera with a miniature lens. The camera may include, for example, a charge-coupled device (CCD) or a complementary metal-oxide-semiconductor (CMOS) image sensor with a few millions or tens of millions of pixels. When implemented using a pinhole camera, the camera may have nearly infinite depth of field such that everything appears in focus without lens distortion, and thus no focusing may be needed. In some embodiments, I/O devices 1440 may include a location tracking device, such as a global positioning system (GPS) receiver. In some embodiments, I/O devices 1440 may include a wired communication port, such as a micro-USB, Lightning, or Thunderbolt transceiver.

I/O devices 1440 may also include, for example, a speaker, a media player, a display device, a communication port, or the like. For example, I/O devices 1440 may include a display device, such as an LED or LCD display and the corresponding driver circuit. I/O devices 1440 may include a text, audio, or video player that may display a text message, play an audio clip, or display a video clip.

Computing device 1400 may include a power device 1460, such as a rechargeable battery for providing electrical power to other circuits on computing system 1400. The rechargeable battery may include, for example, one or more alkaline batteries, lead-acid batteries, lithium-ion batteries, zinc-carbon batteries, and NiCd or NiMH batteries. Computing device 1400 may also include a battery charger for charging the rechargeable battery. In some embodiments, the battery charger may include a wireless charging antenna that may support, for example, one of Qi, Power Matters Association (PMA), or Association for Wireless Power (A4WP) standard, and may operate at different frequencies. In some embodiments, the battery charger may include a hard-wired connector, such as, for example, a micro-USB or Lightning® connector, for charging the rechargeable battery using a hard-wired connection. Power device 1460 may also include some power management integrated circuits, power regulators, power convertors, and the like.

In some embodiments, computing system 1400 may include one or more sensors 1470. Sensors 1470 may include, for example, a passive infrared motion sensor as described above or an active motion sensor. The active motion sensor may include, for example, an infrared active motion sensor or an acoustic motion sensor that actively transmits infrared or acoustic signals and detects reflected signals. Sensors 1470 may also include, for example, a temperature sensor, an ambient light sensor, a barometer, an accelerometer, a sound level meter, or any other similar module operable to provide sensory output and/or receive sensory input. For example, an ambient light sensor may be used to sense ambient light to automatically turn on or off the motion sensor.

Computing device 1400 may be implemented in many different ways. In some embodiments, the different components of computing system 1400 described above may be integrated to a same printed circuit board. In some embodiments, the different components of computing system 1400 described above may be placed in different physical locations and interconnected by, for example, electrical wires. Computing device 1400 may be implemented in various physical forms and may have various external appearances. The components of computing system 1400 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer-readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiments shown herein but is to be accorded the widest scope consistent with the following claims and the principles and novel features disclosed herein.

What is claimed is:

1. A computer-implemented method comprising:
obtaining movement data associated with a subject and measured by a stationary motion sensor, wherein:
the movement data includes a series of values representing a series of movement events of the subject crossing fields of view of the stationary motion sensor; and
each movement event in the series of movement events is associated with a respective time stamp;
extracting a plurality of features from the movement data;
determining, using a machine-learning model and based upon the plurality of features, that the movement data is consistent with symptoms of an illness; and
generating an output indicating a result of the determination.

2. The computer-implemented method of claim 1, wherein extracting the plurality of features from the movement data comprises:
generating, based on the time stamps, activity data including, for each pair of adjacent movement events in the series of movement events, a respective inverse value of a time interval between the pair of adjacent movement events; and
extracting a set of features from the activity data, wherein the plurality of features includes the set of features extracted from the activity data.

3. The computer-implemented method of claim 2, wherein the set of features includes at least one of a shape or a scale of a Generalized Pareto Distribution of the activity data.

4. The computer-implemented method of claim 2, wherein the set of features includes at least one of:
multiscale entropies for a plurality of different time scales of the activity data;
a mean of the multiscale entropies; or
a variance of the multiscale entropies.

5. The computer-implemented method of claim 1, wherein extracting the plurality of features from the movement data comprises:
generating, based on the time stamps, time difference data indicative time intervals between adjacent movement events in the series of movement events; and
computing a set of statistical parameters of the time difference data, the plurality of features including the set of statistical parameters of the time difference data.

6. The computer-implemented method of claim 5, wherein the set of statistical parameters of the time difference data includes at least one of a mean, variance, skewness, kurtosis, or interquartile range of the time difference data.

7. The computer-implemented method of claim 1, wherein determining that the movement data is consistent with the symptoms of the illness includes at least one of:
normalizing the plurality of features;
performing a forward feature selection-based classification; or
estimating an illness severity value or a confidence level of the determination.

8. The computer-implemented method of claim 1, wherein the machine-learning model includes one or more binary classifiers.

9. The computer-implemented method of claim 1, wherein the machine-learning model includes a logistic regression model.

10. The computer-implemented method of claim 1, wherein the stationary motion sensor includes a passive infrared sensor.

11. The computer-implemented method of claim 10, wherein the passive infrared sensor includes a pyroelectric infrared sensor.

12. The computer-implemented method of claim 1, wherein the movement data is collected at a pre-set sampling frequency.

13. A computer-program product tangibly embodied in a non-transitory machine-readable storage medium, including instructions configured to cause one or more data processors to perform operations including:
obtaining movement data associated with a subject and measured by a stationary motion sensor, wherein:
the movement data includes a series of values representing a series of movement events of the subject crossing fields of view of the stationary motion sensor; and
each movement event in the series of movement events is associated with a respective time stamp;
extracting a plurality of features from the movement data;
determining, using a machine-learning model and based upon the plurality of features, that the movement data is consistent with symptoms of an illness; and
generating an output indicating a result of the determination.

14. The computer-program product of claim 13, wherein extracting the plurality of features from the movement data comprises:
generating, based on the time stamps, activity data including, for each pair of adjacent movement events in the series of movement events, a respective inverse value of a time interval between the pair of adjacent movement events; and
extracting a set of features from the activity data, wherein the plurality of features includes the set of features extracted from the activity data.

15. The computer-program product of claim 14, wherein the set of features includes at least one of:
a shape of a Generalized Pareto Distribution of the activity data;
a scale of the Generalized Pareto Distribution of the activity data;
multiscale entropies for a plurality of different time scales of the activity data;
a mean of the multiscale entropies; or
a variance of the multiscale entropies.

16. The computer-program product of claim 13, wherein extracting the plurality of features from the movement data comprises:
generating, based on the time stamps, time difference data indicative time intervals between adjacent movement events in the series of movement events; and
computing a set of statistical parameters of the time difference data, the plurality of features including the set of statistical parameters of the time difference data,
wherein the set of statistical parameters of the time difference data includes at least one of a mean, variance, skewness, kurtosis, or interquartile range of the time difference data.

17. A system comprising:
one or more data processors; and
a non-transitory computer-readable storage medium containing instructions which, when executed on the one or more data processors, cause the one or more data processors to perform operations including:
obtaining movement data associated with a subject and measured by a stationary motion sensor, wherein:
the movement data includes a series of values representing a series of movement events of the subject crossing fields of view of the stationary motion sensor; and
each movement event in the series of movement events is associated with a respective time stamp;
extracting a plurality of features from the movement data;
determining, using a machine-learning model and based upon the plurality of features, that the movement data is consistent with symptoms of an illness; and
generating an output indicating a result of the determination.

18. The system of claim 17, wherein extracting the plurality of features from the movement data comprises:
generating, based on the time stamps, activity data including, for each pair of adjacent movement events in the series of movement events, a respective inverse value of a time interval between the pair of adjacent movement events; and
extracting a set of features from the activity data, wherein the plurality of features includes the set of features extracted from the activity data.

19. The system of claim 18, wherein the set of features includes at least one of:
a shape of a Generalized Pareto Distribution of the activity data;
a scale of the Generalized Pareto Distribution of the activity data;
multiscale entropies for a plurality of different time scales of the activity data;
a mean of the multiscale entropies; or
a variance of the multiscale entropies.

20. The system of claim 17, wherein extracting the plurality of features from the movement data comprises:
generating, based on the time stamps, time difference data indicative time intervals between adjacent movement events in the series of movement events; and
computing a set of statistical parameters of the time difference data, the plurality of features including the set of statistical parameters of the time difference data,
wherein the set of statistical parameters of the time difference data includes at least one of a mean, variance, skewness, kurtosis, or interquartile range of the time difference data.
